(19) United States
(12) Patent Application Publication
(10) Pub. No.: US 2021/0063391 A1
(43) Pub. Date: Mar. 4, 2021

(54) METHODS AND SYSTEMS FOR BIOCELLULAR MARKER DETECTION AND DIAGNOSIS USING A MICROFLUIDIC PROFILING DEVICE

(71) Applicant: The Regents of the University of Michigan, Ann Arbor, MI (US)

(72) Inventors: Sascha Gooneswardena, Ann Arbor, MI (US); Rahul Rattan, Brighton, MI (US); Robert S. Rosenson, New York, NY (US)

(21) Appl. No.: 17/046,195

(22) PCT Filed: Apr. 8, 2019
(86) PCT No.: PCT/US19/26364

§ 371 Date: Oct. 8, 2020

Related U.S. Application Data
(60) Provisional application No. 62/654,638, filed on Apr. 9, 2018.

(51) Int. Cl. G01N 33/561; G01N 33/50; C12Q 1/6816

(52) U.S. Cl. CPC G01N 33/561; C12Q 2600/158; C12Q 1/6816; G01N 33/5008

(57) ABSTRACT

The present disclosure relates to the use of microfluidic devices and systems to generate dynamic molecular signatures based on the detection of various biocellular markers. In particular, the present disclosure involves generating a dynamic molecular signature or profile using the cells of a subject (e.g., circulating monocytes), for various diagnostic and prognostic purposes, such as characterizing a disease or non-disease state, or predicting drug responsiveness. The microfluidic systems and methods of the present disclosure can be used to rapidly assess a plurality of clinical characteristics, which will ultimately enhance therapeutic efficacy and facilitate disease risk stratification.

### CROSS REFERENCE TO RELATED APPLICATION

This application claims the benefit of U.S. Provisional Patent Application No. 62/654,638, filed Apr. 9, 2018, which is incorporated by reference herein.

### FIELD

The present disclosure relates to the use of microfluidic devices and systems to generate dynamic molecular signatures based on the detection of various biocellular markers. In particular, the present disclosure involves generating a dynamic molecular signature or profile using the cells of a subject (e.g., circulating monocytes), for various diagnostic and prognostic purposes, such as characterizing a disease or non-disease state, or predicting drug responsiveness. The microfluidic systems and methods of the present disclosure can be used to rapidly assess a plurality of clinical characteristics, which will ultimately enhance therapeutic efficacy and facilitate disease risk stratification.

### BACKGROUND

Microfluidic technology has recently been applied to molecular biology and to clinical diagnostics. Because of its small sample requirements and exquisite spatial control, microfluidics-based diagnostics are a valuable complement to existing diagnostic technologies. Studies have suggested that understanding the molecular signatures associated with cellular stress responses can be more informative than static measurements. For example, profiling the activation patterns of circulating immune cells (e.g., monocytes) from clinical patients has been difficult because of the amount and processing of clinical samples required for traditional molecular assays. Microfluidic profiling devices and systems can be used overcome these obstacles with the requisite throughput and fidelity, for example, by facilitating the profiling of various molecular activation patterns of circulating immune cells in parallel with an assessment of markers associated with systemic inflammation and other clinical variables. Dynamic molecular signatures based on biomarkers can also be correlated to the biological effects of various therapeutic agents, which can provide beneficial insight into the relationship between the biomarkers and corresponding clinical assessments of the therapeutic agents.

### SUMMARY

Embodiments of the present disclosure include a method of assessing an effect of a therapeutic agent. In accordance with these embodiments, the method includes: (a) performing an assay on a sample from a subject to detect at least one biocellular marker; and (b) generating a dynamic molecular signature based on the detection of the at least one biocellular marker from the subject; wherein the subject has been administered, or is being administered, at least one therapeutic agent.

Embodiments of the present disclosure also include a method of generating a dynamic molecular signature. In accordance with these embodiments, the method includes: (a) performing an assay on a sample from a subject to detect at least one biocellular marker; and (b) generating a dynamic molecular signature based on the detection of the at least one biocellular marker from the subject; wherein the subject has been administered, or is being administered, at least one therapeutic agent.

In accordance with any of the above embodiments, the assay can be performed using a microfluidic immunoblotting device comprising a flat membrane-contacting surface and a plurality of non-connected parallel microfluidic channels, wherein the entire length of the microfluidic channels are open to the membrane-contacting surface.

In accordance with any of the above embodiments, the assay can be performed by: (a) transferring proteins from a sample onto a membrane; (b) placing a membrane-contacting face of a microfluidic immunoblotting device onto the membrane; (c) injecting an activating buffer into microfluidic channels of the device; (d) injecting primary antibody solutions into the microfluidic channels of the device; (e) detecting binding of antibodies with the antibody solutions to the proteins.

In accordance with any of the above embodiments, the assay can include selecting and isolating a group of cells from the sample prior to generating the dynamic molecular signature.

In accordance with any of the above embodiments, assessing a therapeutic agent can include assessing one or more effects of the therapeutic agent on gene or protein expression or activation.

In accordance with any of the above embodiments, generating a dynamic molecular signature can include quantifying a level of expression or activation of the at least one biocellular marker in the sample from the subject with reference to a control sample.

In accordance with any of the above embodiments, the method can further include correlating the dynamic molecular signature to one or more results of a clinical assessment of the subject.

In accordance with any of the above embodiments, the therapeutic agent can be evolocumab, and the dynamic molecular signature can represent effects of PCSK9 inhibition on the biocellular markers.

In accordance with any of the above embodiments, the at least one biocellular marker can include a protein involved in MAPK, NFkB, Akt, AMPK, mTOR, Jak-STAT, and PKA signaling pathways.

In accordance with any of the above embodiments, the at least one biocellular marker can include at least one of VCAM-1, ICAM-1, LOX-1, MCP-1 and MIP-1α.

In accordance with any of the above embodiments, the at least one biocellular marker can include at least one protein expressed by a peripheral blood mononuclear cell.

In accordance with any of the above embodiments, the at least one biocellular marker can include at least one protein expressed by a circulating monocyte.

In accordance with any of the above embodiments, the method can further include treating the subject with a therapeutic agent based on the dynamic molecular signature.

In accordance with any of the above embodiments, the method can further include altering an aspect of treatment of the subject with the therapeutic agent based on the dynamic molecular signature.

Embodiments of the present disclosure also include a dynamic molecular signature generated from a biological sample from a subject, wherein the signature comprises a detectable level of at least one biocellular marker from the subject across different timepoints or treatment regimens.

In accordance with any of the above embodiments, the at least one biocellular marker comprises a protein involved in MAPK, NFkB, Akt, AMPK, mTOR, Jak-STAT, and PKA signaling pathways. In accordance with any of the above embodiments, the at least one biocellular marker comprises at least one of VCAM-1, ICAM-1, LOX-1, MCP-1 and MIP-1α.

In accordance with any of the above embodiments, the at least one biocellular marker comprises at least one protein expressed by a peripheral blood mononuclear cell. In accordance with any of the above embodiments, the at least one biocellular marker comprises at least one protein expressed by a circulating monocyte.

In accordance with any of the above embodiments, the detectable level of the at least one biocellular marker comprises altered expression or activation of the at least one biocellular marker in the sample from the subject with reference to a control sample.

### BRIEF DESCRIPTION OF THE DRAWINGS

FIG. 1 includes a representative schematic of an immunoblot-based microfluidics device, according to one embodiment of the present disclosure.

FIGS. 2A-2B include representative graphs of inflammation signature quantification for PBMC lysates treated with LPS, Heparinase III and LPS+Heparinase III for 2 hours, according to one embodiment of the present disclosure.

FIGS. 3A-3B include representative graphs of inflammation signature quantification for PBMC lysates treated with Hyaluronidase (Hyal), Neuraminidase (Neu) and Spironolactone for 2 hours, according to one embodiment of the present disclosure.

FIGS. 4A-4B include representative graphs of inflammation signature quantification for PBMC lysates treated with LPS, Hyaluronidase and LPS+Hyaluronidase for 2 hours, according to one embodiment of the present disclosure.

### DEFINITIONS

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

As used herein and in the appended claims, the singular forms “a”, “an” and “the” include plural reference unless the context clearly dictates otherwise. Thus, for example, reference to “a device” is a reference to one or more devices and equivalents thereof known to those skilled in the art, and so forth.

As used herein, the term “and/or” includes any and all combinations of listed items, including any of the listed items individually. For example, “A, B, and/or C” encompasses A, B, C, AB, AC, BC, and ABC, each of which is to be considered separately described by the statement “A, B, and/or C.”

As used herein, the term “comprise” and linguistic variations thereof denote the presence of recited feature(s), element(s), method step(s), etc. without the exclusion of the presence of additional feature(s), element(s), method step(s), etc. Conversely, the term “consisting of” and linguistic variations thereof, denotes the presence of recited feature(s), element(s), method step(s), etc. and excludes any unrecited feature(s), element(s), method step(s), etc., except for ordinarily-associated impurities. The phrase “consisting essentially of” denotes the recited feature(s), element(s), method step(s), etc. and any additional feature(s), element(s), method step(s), etc. that do not materially affect the basic nature of the composition, system, or method. Many embodiments herein are described using open “comprising” language. Such embodiments encompass multiple closed “consisting of” and/or “consisting essentially of” embodiments, which may alternatively be claimed or described using such language.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

As used herein, the term “antibody” includes covers monoclonal antibodies, polyclonal antibodies, dimers, multimers, multispecific antibodies (e.g., bispecific antibodies), and antibody fragments, so long as they exhibit the desired biological activity (Miller et al (2003) Jour. of Immunology 170:4854-4861; herein incorporated by reference in its entirety). Antibodies may be murine, human, humanized, chimeric, or derived from other species. An antibody is a protein generated by the immune system that is capable of recognizing and binding to a specific antigen. (Janeway, C., Travers, P., Walport, M., Shlomchik (2001) Immuno Biology, 5th Ed., Garland Publishing, New York; herein incorporated by reference in its entirety). A target antigen generally has numerous binding sites, also called epitopes, recognized by CDRs on multiple antibodies. Each antibody that specifically binds to a different epitope has a different structure. Thus, one antigen may have more than one corresponding antibody. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g., IgG, IgE, IgM, IgD, and IgA), class (e.g., IgG1, IgG2, IgG3, IgG4, IgA1 and IgA2) or subclass of immunoglobulin molecule. The immunoglobulins can be derived from any species, including human, murine, or rabbit origin.

As used herein, the term “antibody fragment” refers to a portion of a full-length antibody, generally the antigen binding or variable region thereof. Examples of antibody fragments include Fab, Fab′, F(ab′)₂, and Fv fragments; diabodies; linear antibodies; fragments produced by a Fab expression library, anti-idiotypic (anti-Id) antibodies, CDR (complementary determining region), and epitope-binding fragments of any of the above which immunospecifically bind to antigens.

As used herein, the phrase “multiplex” or grammatical equivalents refers to the detection, analysis or amplification of more than one target sequence of interest. In one embodiment multiplex refers to >3, >5, >8, >10, >20>50, >100, etc. “Multiplexability” refers to the quality of a particular device, reagent, system, platform, kit, etc. to be used in a multiplex fashion.

As used herein, the term “sample” is used in its broadest sense. In one sense, it is meant to include cells (e.g., human, bacterial, yeast, and fungi), an organism, a specimen or culture obtained from any source, as well as biological and environmental samples. Biological samples may be obtained from animals (including humans) and refers to a biological material or compositions found therein, including, but not limited to, bone marrow, blood, serum, platelet, plasma, interstitial fluid, urine, cerebrospinal fluid, nucleic acid, DNA, tissue, and purified or filtered forms thereof. Environmental samples include environmental material such as surface matter, soil, water, crystals and industrial samples. Such examples are not however to be construed as limiting the sample types applicable to the present invention.

As used herein, the terms “component,” “components,” or “at least one component,” refer generally to a capture antibody, a detection or conjugate a calibrator, a control, a sensitivity panel, a container, a buffer, a diluent, a salt, an enzyme, a co-factor for an enzyme, a detection reagent, a pretreatment reagent/solution, a substrate (e.g., as a solution), a stop solution, and the like that are included in a kit for assay of a test sample, such as a patient urine, whole blood, serum or plasma sample, in accordance with the methods described herein and other methods known in the art. Some components are in solution or lyophilized for reconstitution for use in an assay and are included as part of a kit.

As used herein the terms “label” and “detectable label” generally refer to a moiety attached to an antibody or an analyte to render the reaction between the antibody and the analyte detectable, and the antibody or analyte so labeled is referred to as “detectably labeled.” A label can produce a signal that is detectable by visual or instrumental means. Various labels include signal-producing substances, such as chromagens, fluorescent compounds, chemiluminescent compounds, radioactive compounds, and the like. Representative examples of labels include moieties that produce light (e.g., acridinium compounds), and moieties that produce fluorescence (e.g., fluorescein). Other labels are described herein. In this regard, the moiety, itself, may not be detectable but may become detectable upon reaction with yet another moiety. Use of the term “detectably labeled” is intended to encompass such labeling.

“Risk assessment,” “risk classification,” “risk identification,” or “risk stratification” of subjects (e.g., patients) as used herein refers to the evaluation of factors including biomarkers, to predict the risk of occurrence of future events including disease onset or disease progression and management of administered therapeutic agents, so that treatment decisions regarding the subject may be made on a more informed basis.

“Subject” and “patient” as used herein interchangeably refers to any vertebrate, including, but not limited to, a mammal and a human. In some embodiments, the subject may be a human or a non-human. The subject or patient may be undergoing various forms of treatment.

“Mammal” as used herein refers to any member of the class Mammalia, including, without limitation, humans and nonhuman primates such as chimpanzees and other apes and monkey species; farm animals such as cattle, sheep, pigs, goats, llamas, camels, and horses; domestic mammals such as dogs and cats; laboratory animals including rodents such as mice, rats, rabbits, guinea pigs, and the like. The term does not denote a particular age or sex. Thus, adult and newborn subjects, as well as fetuses, whether male or female, are intended to be included within the scope of this term.

“Treat,” “treating” or “treatment” are each used interchangeably herein to describe reversing, alleviating, or inhibiting the progress of a disease and/or injury, or one or more symptoms of such disease, to which such term applies. Depending on the condition of the subject, the term also refers to preventing a disease, and includes preventing the onset of a disease, or preventing the symptoms associated with a disease. A treatment may be either performed in an acute or chronic way. The term also refers to reducing the severity of a disease or symptoms associated with such disease prior to affliction with the disease. Such prevention or reduction of the severity of a disease prior to affliction refers to administration of a pharmaceutical composition to a subject that is not at the time of administration afflicted with the disease. “Preventing” also refers to preventing the recurrence of a disease or of one or more symptoms associated with such disease. “Treatment” and “therapeutically,” refer to the act of treating, as “treating” is defined above.

As used herein, the terms “biomarker,” and “biocellular marker” generally refer to a biochemical (e.g., nucleic acids, amino acids, lipids, hormones, metabolites, etc.) that is identified, detected, measured, and/or quantified, which is used individually or in combination to make a diagnostic determination, including but not limited to, a diagnostic determination pertaining to a disease state and/or efficacy of a therapeutic agent.

As used herein, the terms “molecular signature,” “dynamic molecular signature,” “proteomic profile,” and “molecular profile” generally refer to one or more data points pertaining to the expression, activation, and/or location of at least one biomarker or biocellular marker that is used to characterize a disease state of a sample or a subject and/or to make a diagnostic determination regarding the efficacy of a therapeutic agent.

As used herein, the term “therapeutic agent” generally refer to agents capable of having a therapeutic effect in a subject, including but not limited to, antimicrobial agents, antiparasitic agents, antibiotics, antihistamines, decongestants, antipruritics, antimetabolites, antiglaucoma agents, anti-cancer agents, antiviral agents, antifungal agents, antimycotics, anti-inflammatory agents, anti-diabetic agents, anesthetic agents, anti-depressant agents, analgesics, anti-coagulants, ophthalmic agents, angiogenic factors, immunosuppressants, anti-allergic agents, spermicides, humectants and emollients, hormones, and any derivatives or variants thereof.

Unless otherwise defined herein, scientific and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry and hybridization described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

### DETAILED DESCRIPTION

The present disclosure relates to the use of microfluidic devices and systems to generate dynamic molecular signatures based on the detection of various biocellular markers. In particular, the present disclosure involves generating a dynamic molecular signature or profile using the cells of a subject (e.g., circulating monocytes), for various diagnostic and prognostic purposes, such as characterizing a disease or non-disease state, or predicting drug responsiveness. The microfluidic systems and methods of the present disclosure can be used to rapidly assess a plurality of clinical characteristics, which will ultimately enhance therapeutic efficacy and facilitate disease risk stratification.

Although inflammation is known to be a driver of a diverse spectrum of chronic diseases, profiling circulating blood biomarkers (e.g., CRP, cytokines, lipoproteins) has not greatly added to current management strategies. One possible explanation for this is that circulating biomarkers capture the systemic inflammatory state of a patient rather than the local inflammation in specific organs that drives chronic diseases. In animal models, the innate immune system responds to local inflammatory stimuli, leading to the recruitment of circulating monocytes which respond to these perturbations. As sentinels of innate immune system, the circulating monocytes provide a snapshot into organ-specific microenvironments providing a functional “biocellular-marker” that integrates both the systemic and local inflammatory state of patients with chronic diseases including cardiovascular disease, Alzheimer's, rheumatalogic diseases, and chronic lung disease.

Using a microfluidic platform, methods and systems were developed to probe the dynamic molecular signatures associated with inflammatory stimulation of patient peripheral blood mononuclear cells (PBMCs) that greatly improves the diagnostic yield compared with static blood biomarkers in patients with chronic diseases. One approach includes the stimulation of patient-specific PBMCs with various toll-like receptor (TLR) ligands and examining the molecular activation using a microfluidic platform. This has implications for not only disease biomarkers, but also drug discovery and translational medicine applications. Assessing protein signatures in patient PBMCs and other circulating cells and how these cells may respond to inflammatory stimuli has not previously been possible because of the large requirements of cells and the variability in traditional probing methodologies.

Section headings as used in this section and the entire disclosure herein are merely for organizational purposes and are not intended to be limiting.

### 1. Microfluidic Profiling Devices and Systems

Microfluidic technology has recently been applied to various aspects of biomedical research, clinical diagnostics, and molecular therapeutics. Because of its small sample requirements and exquisite spatial control, microfluidics can enhance existing diagnostic technologies. For example, studies have suggested that understanding the molecular signatures associated immune cell stress responses can be more informative than static measures of inflammation (e.g., immunoassays such as ELISAs). However, profiling the activation patterns of circulating monocytes in a clinical context has been difficult because of the amount and processing of clinical samples required for traditional molecular assays.

The microfluidic profiling devices, methods, and systems of the present disclosure overcome many of the obstacles associated with current diagnostic methods involving immune cells. For example, the microfluidic profiling devices, methods, and systems of the present disclosure facilitate the profiling of the molecular activation patterns of circulating blood monocytes in parallel with biomarkers of systemic inflammation, lipoproteins, and other clinical metrics with enhanced throughput and fidelity. In this manner, dynamic molecular signatures can be generated from patient samples and used to inform and/or refine treatment regimens.

In some embodiments, the microfluidic profiling devices, methods, and systems of the present disclosure can be used to identify the biological effects of PCSK9 inhibition on immune cell function, for example, providing mechanistic insight into the relationship between lipoprotein metabolism, microvascular dysfunction, and inflammation. The generation of novel dynamic molecular signatures can result in identification of “at-risk” populations that would benefit from more aggressive LDL cholesterol reduction.

In some embodiments, microfluidic profiling devices, methods, and systems of the present disclosure can be used to profile monocytes in patients, and can be used in conjunction with the use of flow cytometry. Monocytes are key innate immune system mediators of inflammatory responses and have been implicated as drivers of CVD. Monocyte subsets are characterized using flow cytometry for surface expression of FcγIII receptor CD16 and the lipopolysaccharide receptor CD14. In some embodiments, blood is be collected in Cell Preparation Tubes (CPT) with sodium citrate (BD, New Jersey). Cells are resuspended in FACS buffer and stained with fluorescently labeled antibodies. Monocyte subsets are defined using CD14 and CD16 biomarkers. The Nomenclature Committee of the International Union of Immunological Societies defines three monocyte subsets by this method: CD14++CD16− (classical), CD14++CD16+ (intermediate), and CD14+CD16++ (non-classical). Flow cytometric analysis is performed on a BD Accuri C6 flow cytometer and the data analyzed using FlowJo software (Tree Star, Inc). Monocyte subsets are defined using CD14 and CD16 biomarkers. Because cholesterol metabolism has been associated with monocyte egress from the bone marrow, PCSK9 inhibition can alter monocyte subsets compared with patients not on therapy.

Functional interactions between clinical data, cell-surface markers, and molecular activation of signaling pathways can be systematically defined using the microfluidic proteomic platforms, systems, and methods of the present disclosure, which include the use of PCR arrays. Briefly, circulating monocytes are isolated and processed as described above. To understand how PCSK9 inhibition modulates transcriptional and signaling responses to inflammatory stimuli, monocytes are stimulated with TLR ligands and RNA and protein is isolated at various timepoints, including, for example, two timepoints (e.g., 30 min and 2 hours). Inflammatory transcriptional programs are profiled using PCR arrays (SA Biosciences, Maryland). Activation of inflammatory signaling pathways (e.g., MAPK, NFkB, Akt, AMPK, mTOR, Jak-STAT, PKA) are profiled using the microfluidic immunoblotting platform and methods of the present disclosure.

It is likely that PCSK9 inhibition significantly alters TLR activation of signaling pathways and inflammatory transcriptional programs in monocytes, which are captured in dynamic molecular signatures generated using the microfluidic immunoblotting platform, systems, and methods of the present disclosure. These dynamic responses characteristic of circulating monocytes are not captured with typical profiling systems and methods (e.g., immunoassays such as ELISAs); by using circulating monocytes as “biocellular” markers of inflammatory risk, higher content profiling is generated and used to identify how PCSK9 modulates inflammatory responses. Additionally, these dynamic molecular signatures are used to identify patients most at risk of CVD disease and those who would benefit from more aggressive LDL cholesterol reduction treatments and intervention.

Embodiments of the present disclosure also include methods of assessing circulating biomarkers that interface with inflammation and endothelial function using the microfluidic immunoblotting platforms, systems, and methods described herein. These include, but are not limited to, soluble VCAM-1, ICAM-1, LOX-1, MCP-1 and MIP-1α. Due to the critical role of low wall shear stress in endothelial dysfunction, inflammation and site-specificity of atherosclerosis, blood viscosity profiles can also be assessed. Lipoprotein subclasses are measured by NMR spectroscopy (LabCorp), based on the finding that there is an inverse association between large HDL particle concentration and myocardial perfusion reserve index.

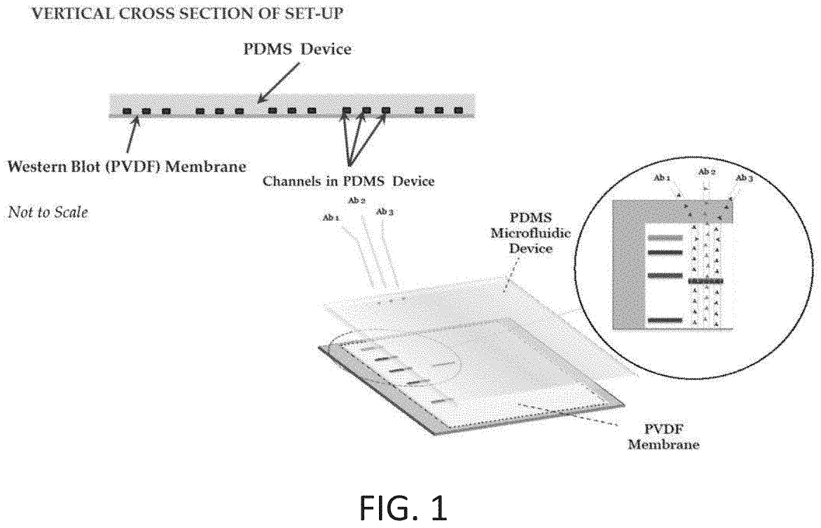

As shown in FIG. 1, the microfluidic immunoblotting systems and devices of the present disclosure allow for targeted, high-throughput proteomic assessment of signaling pathways in individual patient samples. Embodiments of a microfluidic-proteomic device and system were recently filed in PCT Application No. PCT/US14/24684, U.S. patent application Ser. No. 14/774,578, and U.S. Provisional Patent Application Ser. No. 61/777,682, all of which are herein incorporated by reference in their entirety and for all purposes.

Embodiments of the present disclosure include microfluidic devices, systems, and methods for use in immunoblotting applications. In particular, devices and methods provided herein have the advantages of traditional Western blotting with increased throughput and multiplexability, and decreased time, sample, and reagent requirements. While traditional Western blotting can yield important information (e.g., about the interaction between a biological system (e.g., humans) and an outside stimulus (e.g., an anti-inflammatory drug)), they are expensive, both in terms of reagents and time. In certain embodiments, the present disclosure provides devices and methods that provide similar and/or greater information content as traditional Western blotting, but with decreased reagent and time consumption, and increased throughput and multiplexability. In some embodiments, the present disclosure provides a microfluidic device comprising sets of microfluidic channels configured to overlay the lanes present on a protein-containing membrane (FIG. 1). In various embodiments, proteins on the membrane are probed by antibodies within the microfluidic channels (FIG. 1). In some embodiments, a single protein lane on a membrane is overlayed by two or more microfluidic channels (e.g., 2, 3, 4, 5, 6, 7, 8, 9, 10, or more), each containing a different antibody for probing the protein lane (FIG. 1).

In some embodiments, the present disclosure provides numerous advantages over traditional immunoblotting. For example, instead of incubating the entire membrane with a single type of primary antibody, a much smaller quantity of antibody is provided in a single microfluidic channel per protein lane. In embodiments in which multiple microfluidic channels are provided per protein lane, a single protein lane may be probed by multiple different antibodies simultaneously. In some embodiments, the present disclosure allows for probing with more types of antibodies (e.g., 2 . . . 5 . . . 10 . . . 20, or more per lane of a gel) using less reagent (e.g., microfluidic channel vs. incubating the entire membrane), less sample (e.g., a single lane on a gel vs. a different gel for each different antibody), and less time (e.g., a single gel and membrane transfer vs. a different gel and transfer) than traditional immunoblotting. In some embodiments, from a time perspective, devices and method of the present disclosure can complete 3, 5, 10, 20, or more immunoblots in the time it takes to complete one traditional Western blot. In some embodiments, from a cost perspective, devices and methods of the present disclosure can complete an immunoblot for less than 1% of the cost of a traditional Western Blot.

a. Device/Systems

In certain embodiments, microfluidic devices are provided that comprises one or more sets (e.g., 1, 2, 3, 4, 5, 6, 7, 8, 9, 10, 11, 12, 13, 14, 15, 16, 17, 18, 19, 20, or more) of one or more microfluidic channels (e.g., 1, 2, 3, 4, 5, 6, 7, 8, 9, 10, 11, 12, 13, 14, 15, 16, 17, 18, 19, 20, or more). In some embodiments, the channels run along the flat surface of the device, such that fluid contained within the channels is exposed to the surface of the device. In some embodiments, each channel comprises a reservoir or other fluid-introduction means for introduction of antibody solution into the microfluidic channel.

In some embodiments, a microfluidic device does not require valves, pumping, mixing chambers, and/or other more complex elements that are common to microfluidic devices, but increase the cost of a device and the complexity of its use. In some embodiments, a microfluidic device does not require integration with gel electrophoresis in order to carry out immunoblotting.

In some embodiments, a microfluidic device comprises one or more sets of channels, each containing one or more channels. Each set is configured to probe a single lane on of a protein gel. In some embodiments, each channel of a set is configured to probe the protein lane with a different antibody. Each channel set may comprise the same or different number of channels.

In some embodiments, the device may be of any suitable size and dimensions. In some embodiments, a device is configured to match the dimensions of a particular gel or membrane. In some embodiments, a device is configured to be usable with gels and membranes of different sizes and/or dimensions. In some embodiments, devices comprise a footprint (e.g., membrane-contacting face) with dimensions between 1 cm and 50 cm or more (e.g., 1 cm×1 cm, 4 cm×6 cm, 8 cm×6 cm, 5 cm×20 cm, 20 cm×40 cm, etc.). In some embodiments, the length and/or width of the membrane-contacting face of a microfluidic device is 1 cm . . . 2 cm . . . 3 cm . . . 4 cm . . . 5 cm . . . 6 cm . . . 7 cm . . . 8 cm . . . 9 cm . . . 10 cm . . . 11 cm . . . 12 cm . . . 13 cm . . . 14 cm . . . 15 cm . . . 16 cm . . . 17 cm . . . 18 cm . . . 19 cm . . . 20 cm . . . 21 cm . . . 22 cm . . . 23 cm . . . 24 cm . . . 25 cm . . . 26 cm . . . 27 cm . . . 28 cm . . . 29 cm . . . 30 cm . . . 31 cm . . . 32 cm . . . 33 cm . . . 34 cm . . . 35 cm . . . 36 cm . . . 37 cm . . . 38 cm . . . 39 cm . . . 40 cm . . . 41 cm . . . 42 cm . . . 43 cm . . . 44 cm . . . 45 cm . . . 46 cm . . . 47 cm . . . 48 cm . . . 49 cm . . . 50 cm.

In some embodiments, channels of a microfluidic device may be of any suitable size and dimensions. In some embodiments, channels are so dimensioned to allow the appropriate solutions to efficiently traverse and/or occupy the microfluidic channels. In some embodiments, the length of a channel is proportional to the length of the device. In some embodiments, the length of a channel is proportional to the length of the membrane and/or gel with which it is configured for use. In some embodiments, channels are between 1 cm and 50 cm in length (e.g., 1 cm . . . 2 cm . . . 3 cm . . . 4 cm . . . 5 cm . . . 6 cm . . . 7 cm . . . 8 cm . . . 9 cm . . . 10 cm . . . 11 cm . . . 12 cm . . . 13 cm . . . 14 cm . . . 15 cm . . . 16 cm . . . 17 cm . . . 18 cm . . . 19 cm . . . 20 cm . . . 21 cm . . . 22 cm . . . 23 cm . . . 24 cm . . . 25 cm . . . 26 cm . . . 27 cm . . . 28 cm . . . 29 cm . . . 30 cm . . . 31 cm . . . 32 cm . . . 33 cm . . . 34 cm . . . 35 cm . . . 36 cm . . . 37 cm . . . 38 cm . . . 39 cm . . . 40 cm . . . 41 cm . . . 42 cm . . . 43 cm . . . 44 cm . . . 45 cm . . . 46 cm . . . 47 cm . . . 48 cm . . . 49 cm . . . 50 cm). In some embodiments, a channel is of appropriate width and/or depth to allow the necessary solutions (e.g., antibody solutions) to flow/traverse, occupy, and/or fill the channel. In some embodiments, the width of a channel is optimized to provide the desired number of channels in a set of a particular width. In some embodiments, as channels are narrowed to accommodate more lanes per set, channels are deepened to provide the necessary channel volumes. In some embodiments, a set of channels ranges in width from 1 mm to 50 cm (e.g., 1 mm . . . 2 mm . . . 3 mm . . . 4 mm . . . 5 mm . . . 6 mm . . . 7 mm . . . 8 m . . . 9 mm . . . 1 cm . . . 2 cm . . . 3 cm . . . 4 cm . . . 5 cm . . . 6 cm . . . 7 cm . . . 8 cm . . . 9 cm . . . 10 cm . . . 11 cm . . . 12 cm . . . 13 cm . . . 14 cm . . . 15 cm . . . 16 cm . . . 17 cm . . . 18 cm . . . 19 cm . . . 20 cm . . . 21 cm . . . 22 cm . . . 23 cm . . . 24 cm . . . 25 cm . . . 26 cm . . . 27 cm . . . 28 cm . . . 29 cm . . . 30 cm . . . 31 cm . . . 32 cm . . . 33 cm . . . 34 cm . . . 35 cm . . . 36 cm . . . 37 cm . . . 38 cm . . . 39 cm . . . 40 cm . . . 41 cm . . . 42 cm . . . 43 cm . . . 44 cm . . . 45 cm . . . 46 cm . . . 47 cm . . . 48 cm . . . 49 cm . . . 50 cm). In some embodiments, set width is configured to match the lane width of a gel and/or membrane. In some embodiments, a set of microchannels comprises one or more channels (e.g., 1, 2, 3, 4, 5, 6, 7, 8, 9, 10 . . . 20 . . . 30 . . . 40 . . . 50 . . . 100 . . . 500, etc.). In some embodiments, the number of channels per set is dependent upon the width of the set, the spacing between channels, and the width of the channels (e.g., which may be dependent upon the type of solution to be used within the channels). In some embodiments, channels widths range from 50 μm to 500 μm (e.g., 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm . . . 250 μm . . . 300 μm . . . 350 μm . . . 400 μm . . . 450 μm . . . 500 μm). In some embodiments, channel widths range from 100 μm to 200 μm (e.g., 125 μm to 175 μm, 140 μm to 160 μm, about 150 μm, 150 μm). In some embodiments, channel depths range from 25 μm to 200 μm (e.g., 25 μm, 30 μm, 40 μm, 50 μm, 60 μm, 70 μm, 80 μm, 90 μm, 100 μm, 110 μm, 120 μm, 130 μm, 140 μm, 150 μm, 160 μm, 170 μm, 180 μm, 190 μm, 200 μm). In some embodiments, channel widths range from 75 μm to 125 μm (e.g., 90 μm to 110 μm, about 100 μm, 100 μm). In some embodiments, channels accommodate between 0.1 μl and 5 μl of solution per channel (e.g., 0.1 μl, 0.2 μl, 0.3 μl, 0.4 μl, 0.5 μl, 0.6 μl, 0.7 μl, 0.8 μl, 0.9 μl, 1.0 μl, 1.1 μl, 1.2 μl, 1.3 μl, 1.4 μl, 1.5 μl, 1.6 μl, 1.7 μl, 1.8 μl, 1.9 μl, 2 μl . . . 3 μl . . . 4 μl . . . 5 μl).

In some embodiments, microfluidic devices are provided as part of a kit along with one or more of appropriately sized membranes, appropriately sized gel with lanes configured to match channels of the device, antibody solutions, buffer, activation solution, comb for pouring gels with lanes configured to match channels of the device, instructions, etc. In some embodiments, a device or kit is optimized for a particular assay. In some embodiments, a kit is provided with antibodies and lane configuration for performing a specific assay.

In some embodiments, the channel sets and/or microfluidic channels are not consistent across the device. Channels on a single device may vary across a single device according to one or more of set width, number of microchannels per set, microchannel width, spacing, etc.

b. Methods

In some embodiments, devices of the present disclosure are utilized to perform immunoblot experiments, using any suitable techniques and reagents.

An exemplary procedure for immunoblotting with a device of the present disclosure is as follows. A protein-containing sample is loaded into the wells of a gel (e.g., polyacrylamide gel) and proteins present in the sample are separated by gel electrophoresis (e.g., native gel electrophoresis, SDS-PAGE, etc.). A membrane (e.g., PVDF membrane) is placed atop the gel to allow transfer of the proteins onto the membrane. The membrane is then placed on a glass slide. A microfluidic device is placed on top of membrane, and the microfluidic channels are aligned with the protein lanes. Blocking buffer (e.g., Tween-20 and BSA) is optionally injected in each of the channels. In embodiments with a blocking step, the inlets/outlets of the channels are covered (e.g., with tape) to minimize evaporation during incubation. Antibodies are injected into the appropriate channels (e.g., different antibodies in each microchannel of a set). The inlets/outlets are covered (e.g., with tape) to minimize evaporation during incubation. Following incubation (e.g., 1 hours), the microfluidic device is removed, and the membrane is transferred to a solution of an appropriate secondary antibody (e.g., the membrane is left in this solution for 1 hour, or until the membrane is completely wet). The membrane is removed from the secondary antibody solution and washed with fresh blocking buffer (e.g., 3 minutes, 5 times each). The membrane is transferred to a developing solution (e.g., 30 minutes), and the membrane is removed and rinsed with DI water. The membrane is then allowed to dry. In some embodiments, any or all of the above steps may be altered for an alternative procedure or to accommodate a specific use. For example, in some embodiments, the device is placed onto the membrane with antibodies pre-loaded into the channels. In other embodiments, the blocking, secondary antibody solution, washing, and developing solutions are applied through the microfluidic channels. In some embodiments, a sample is placed on a membrane directly or from a surface other than a gel (e.g., microtiter plate, array, etc.).

In some embodiments, the same sample under the same conditions is run on multiple wells of a gel. In such cases, the microfluidic channels of multiple sets of channels may all be loaded with different antibodies to interrogate the same sample. In other embodiments, different samples (or the same sample under different conditions) are run on two or more lanes of a gel. In such cases, the various channel sets may be loaded identically, to probe each sample with the same set of antibodies.

In some embodiments, the device and/or membrane comprise hydrophobic surfaces (e.g., PDMS and PVDF). Experiments conducted during development of embodiments of the present disclosure revealed that it is challenging to achieve a suitable seal between the device and membrane. In some embodiments, to overcome this challenge, an activation step (e.g., of the device and/or of the membrane) is performed to enhance the seal between the device and membrane. In some embodiments, the seal is activated through the microfluidic channels. In some embodiments, an activating solution is applied to the membrane-contacting surface of the device. In some embodiments, an activating solution is applied to the membrane. In some embodiments, the present invention provides systems (e.g., assays) comprising a device of the present invention and a membrane. In some embodiments, an activating solution is located or placed between said device and said membrane. In some embodiments, the activating solution comprises polysorbate-20 (Tween-20) and BSA. In some embodiments, the activating solution comprises 0.01-0.5% Tween-20 and 0.01-0.5% BSA. In some embodiments, the activating solution comprises 0.05-0.15% Tween-20 and 0.05-0.15% BSA. In some embodiments, the activating solution comprises 0.1% Tween-20 and 0.1% BSA. In some embodiments, the membrane comprises membrane nitrocellulose or polyvinylidene fluoride (PVDF). In some embodiments, the membrane comprises peptides, polypeptides, or proteins on or within it. In some embodiments, systems further comprise one or more antibody-containing solutions within the microfluidic channels. In some embodiments, each microfluidic channel of a channel set comprises a different antibody-containing solution. In some embodiments, each channel set comprises the same combination of antibody-containing solutions. In some embodiments, each microfluidic channel comprises a different antibody-containing solution. In some embodiments, the present invention provides methods of immunoblotting comprising applying antibody solutions to a membrane using system of the present invention. In some embodiments, the activation takes advantage of the hydrophobic/hydrophilic differences between the PDMS/PVDF surfaces and the aqueous solutions used.

In some embodiments, solutions are added to microfluidic channels through any suitable structure or method. In some embodiments, solutions are injected into channels. In some embodiments, solutions are injected into reservoirs at one end of the channels. In some embodiments, pumps and/or valves are not required for solution addition. In some embodiments, a needle (e.g., 10-40 gauge (e.g., 12 gauge . . . 16 gauge . . . 20 gauge . . . 24 gauge . . . 27.5 gauge . . . 30 gauge . . . 36 gauge . . . 40 gauge) is used to introduce solutions (e.g., antibody solutions) into the channel. In some embodiments, introduction of solutions from an appropriately sized needle (e.g., 27.5 gauge) into an appropriately sized reservoir produces a “tamponade” effect around the needle that greatly enhances the seal and facilitates filling of the microfluidic channel.

c. Antibodies/Reagents

In some embodiments, solutions for use with the devices described herein are be configured to fill microfluidic channels is an efficient and reproducible manner. To this end, solutions should have appropriate viscosity, hydrophilicity, etc. Experiments were conducted during development of embodiments of the present disclosure to develop solutions with appropriate characteristic for deployment in the microfluidic channels of the devices described herein. In some embodiments, solutions comprise one or more surfactants, detergents, emulsifiers, solubilizers, etc. to provide acceptable/optimal filling the microfluidic channels in a fast and reproducible manner. In some embodiments, solutions comprise one or more of: ammonium lauryl sulfate, sodium lauryl sulfate (SDS, sodium dodecyl sulfate), sodium laureth sulfate, sodium myreth sulfate, dioctyl sodium sulfosuccinate, perfluorooctanesulfonate (PFOS), perfluorobutanesulfonate, linear alkylbenzene sulfonates (LABs), sodium stearate, sodium lauroyl sarcosinate, perfluorononanoate, perfluorooctanoate, alkyltrimethylammonium salts (e.g., cetyl trimethylammonium bromide), cetylpyridinium chloride (CPC), benzalkonium chloride (BAC), benzethonium chloride (BZT), 5-Bromo-5-nitro-1,3-dioxane, dimethyldioctadecylammoniumchloride, cetrimoniumbromide, dioctadecyldimethylammonium bromide (DODAB), CHAPS, cocamidopropyl hydroxysultaine, lecithin, polyoxyethylene glycol alkyl ethers, polyoxypropylene glycol alkyl ethers, glucoside alkyl ethers, polyoxyethylene glycol octylphenol ethers (e.g., Triton X-100), Polyoxyethylene glycol alkylphenol ethers, glycerol alkyl esters, polyoxyethylene glycol sorbitan alkyl esters (e.g., Polysorbate (e.g., Polysorbate 20, Polysorbate 40, Polysorbate 60, Polysorbate 80, etc.)), sorbitan alkyl esters, cocamide MEA, cocamide DEA, dodecyldimethylamine oxide, block copolymers of polyethylene glycol and polypropylene glycol, polyethoxylated tallow amine (POEA), etc.

In some embodiments, solutions (e.g., antibody solutions, activating solutions, blocking solutions, washing solutions, etc.) comprise tailored concentrations of Tween (e.g., Tween-20) and bovine serum albumin (BSA). In some embodiments, useful concentrations of Tween (e.g., Tween-20) and BSA are utilized to provide for efficient flowing of solutions for the length of the microfluidic channels. In some embodiments, useful concentrations of Tween (e.g., Tween-20) and BSA are utilized to provide activation/wetting of the membranes through the microfluidic channels. In some embodiments, solutions comprise between 0.01% and 5% BSA (e.g., 0.01% . . . 0.02% . . . 0.05% . . . 0.1% . . . 0.2% . . . 0.5% . . . 1% . . . 2% . . . 5%). In some embodiments, solutions comprise between 0.01% and 5% Tween (e.g., Tween20) (e.g., 0.01% . . . 0.02% . . . 0.05% . . . 0.1% . . . 0.2% . . . 0.5% . . . 1% . . . 2% . . . 5%). In some embodiments, solutions comprise about 0.1% BSA and about 0.1% Tween20. In some embodiments, solutions comprise 0.1% BSA and 0.1% Tween20.

In some embodiments, solutions (e.g., blocking, activating, washing, antibody, etc.) comprise appropriate salts, buffers, metals, etc. as would be understood by one of skill in the art.

In some embodiments, antibody binding to target proteins are visualized and/or detected through the use of a detectable/observable moieties and/or labels. Suitable labels and/or moieties are detected spectroscopic, photochemical, biochemical, immunochemical, electrical, optical or chemical means. In some embodiments, primary or secondary antibodies are linked to a detection moiety. In some embodiments, detection is performed enzymatically using, for example alkaline phosphatase or horseradish peroxidase. Some embodiments, utilize fluorescent detection. Fluorophores contemplated to be useful in the present disclosure include Alexa dyes (e.g., Alexa 350, Alexa 430, etc.), AMCA, BODIPY 630/650, BODIPY 650/665, BODIPY-FL, BODIPY-R6G, BODIPY-TMR, BODIPY-TRX, Cascade Blue, Cy2, Cy3, Cy5, 6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, Tetramethylrhodamine, Texas Red, etc. One of skill in the art will recognize that these and other detection moieties not mentioned herein can be used with success in various embodiments.

Any antibodies, antibody fragments, or other molecules capable of specifically and stably binding a target, analyte, or antigen find use as the primary and secondary antibodies described herein (e.g., dendrimer, nucleic acid, affinity tag, etc.). Although the embodiments described herein have typically utilized antibodies (e.g., primary antibodies, secondary antibodies) for interrogating the protein contents of a sample, the present disclosure is not limited to the use of antibodies. Embodiments should be read broadly as also encompassing other binding molecules.

### 2. Dynamic Molecular Signatures

Embodiments of the present disclosure are used to generate a dynamic molecular signature corresponding to a variety of disease states and/or clinical characteristics. In accordance with these embodiments, the present disclosure involves generating a dynamic molecular signature or profile using the cells of a subject (e.g., circulating monocytes), for various diagnostic and prognostic purposes, such as characterizing a disease or non-disease state, or predicting drug responsiveness. In some embodiments, systems and methods of the present disclosure include the assessment of cardiovascular diseases (CVDs), including, for example, atherosclerosis. Atherosclerosis is a chronic inflammatory disease of the arterial wall that involves endothelial cell (EC) dysfunction; monocyte recruitment, retention and activation; and vascular smooth muscle cell (SMC) proliferation. ECs are activated by diverse physiologic stimuli including cytokines, catecholamines, and shear stress. Persistent activation of EC can lead to vascular dysfunction and systemic inflammation, both of which are known to drive vascular events including stroke. In aortic endothelial cells and vascular SMCs, NADPH oxidase dependent reactive oxygen species (ROS) increases expression of proprotein convertase subtilisin/kexin type 9 (PCSK9) via lectin LDLR-1 (LOX-1). Atherosclerosis is site specific with increased expression of PCSK9 as in low wall shear stress regions of murine aorta.

Although inflammation is known to be a driver of acute coronary syndromes (ACS) and strokes, profiling circulating inflammatory mediators (e.g., CRP, cytokines, lipoproteins) has not greatly added to current patient management strategies. One possible explanation for this is that circulating inflammatory markers capture the systemic inflammatory state of the patient and not local inflammation in specific arterial beds. In animal models, the innate immune system responds to local plaque rupture, leading to the recruitment of circulating monocytes. In human observational studies, elevated levels of monocytes correlate with infarct and stroke size. Although circulating monocytes are elevated in patients with atherosclerotic diseases, their molecular profiles and how they relate to clinical atherosclerotic cardiovascular disease remain largely unknown. Furthermore, even though it is known that circulating monocytes can change with acute and chronic inflammatory stresses, it remains unclear how PCSK9 inhibition and subacute reductions in lipoproteins impact circulating monocyte subsets and their functional responses. As sentinels of innate immune system, the circulating monocytes may provide a snapshot into local plaque microenvironment providing a functional “biocellular-marker” that integrates both the systemic and local inflammatory state of patients with atherosclerotic disease.

Although experimental models have linked lipoproteins and EC dysfunction with systemic inflammation, relatively little is known about this network in clinical populations and specifically how it changes with PCSK9 inhibition. Because of PCSK9 effects on the LDL receptor, PCSK9 inhibition is likely to inhibit pro-inflammatory changes in circulating monocyte subsets and functions. In circulating monocytes, toll-like receptors (TLR) sense inflammatory molecules. TLR responses are mediated through MyD88 and TRIF, which leads to activation of signaling pathways including Jak-STAT, Akt, AMPK, mTOR, PKA, NFkB, and MAPK, which mediate inflammatory transcriptional programs. The participation of PCSK9 in vascular inflammation is supported by preclinical studies using PCSK9 siRNA in macrophages. These studies have demonstrated that PCSK9 inhibition in macrophages reduces NF-kB inflammatory responses.

Furthermore, oscillatory shear stress increases the activation of pro-inflammatory signaling pathways and reduces activation of anti-inflammatory pathways. The measurement of low shear blood viscosity is used to examine the associations between baseline and on-trial change in low shear blood viscosity with inflammatory mediators. Data on blood viscosity can also be used to investigate the putative role of PCSK9 inhibition on cardiac microvascular dysfunction, as blood flow in the microcirculation is highly dependent on changes in blood viscosity.

Embodiments of the present disclosure find use to generate dynamic molecular signatures corresponding to various cellular signaling pathways, such as those signaling pathways associated with particular disease states. In some embodiments, systems and methods of the present disclosure include assessing PCSK9 inhibition, such as how PCSK9 inhibition alters the circulating monocyte populations, and systematically profiling the transcriptional and proteomic responses to TLRS and how these responses are modulated by PCSK9 inhibition in clinical samples of PBMCs. These molecular signatures can then be correlated with clinical and imaging variables, and can facilitate disease risk stratification.

In accordance with these embodiments, the generation of molecular signatures profiling PCSK9 inhibition enhances understanding of the molecular mechanisms through which PCSK9 and circulating lipoproteins orchestrate TLR response in circulating monocytes. Additionally, PBMC subset analysis and transcriptional and proteomic analysis of inflammatory signaling pathways in response to TLR stimulation can also be performed. Together, these data aid in defining the molecular pathways through which PCSK9 modulates inflammatory responses in circulating monocytes and how these molecular signatures relate to clinical variables known to be important in CVD risk stratification.

### 3. Therapeutic Agents

Embodiments of the present disclosure provide microfluidic immunoblotting systems and methods for generating a dynamic molecular signature or profile of a subject, such as a subject that is receiving treatment with a therapeutic agent or a subject that may be receiving treatment with a therapeutic agent. In some embodiments, the methods include using the dynamic molecular signature of a subject to inform treatment decisions, such as which therapeutic agent to administer to the patient, predicting how a particular subject may respond to a particular therapeutic agent, and altering one or more aspects of a treatment regimen.

As would be recognized by one of ordinary skill in the art based on the present disclosure, the methods and systems described herein can be used to assess the various effects of any therapeutic agent currently used, and any therapeutic agent developed in the future, on a subject based on generation of dynamic molecular signatures, regardless of a particular disease context or disease indication. Therapeutic agents can include, but are not limited to, abarelix, abiraterone acetate, aldesleukin, alemtuzumab, altretamine, anastrozole, asparaginase, bevacizumab, bexarotene, bicalutamide, bleomycin, bortezombib, brentuximab vedotin, busulfan, capecitabine, carboplatin, carmustine, cetuximab, chlorambucil, cisplatin, cladribine, clofarabine, clomifene, crizotinib, cyclophosphamide, dasatinib, daunorubicin liposomal, decitabine, degarelix, denileukin diftitox, denileukin diftitox, denosumab, docetaxel, doxorubicin, doxorubicin liposomal, epirubicin, eribulin mesylate, erlotinib, estramustine, etoposide phosphate, everolimus, exemestane, fludarabine, fluorouracil, fotemustine, fulvestrant, gefitinib, gemcitabine, gemtuzumab ozogamicin, goserelin acetate, histrelin acetate, hydroxyurea, ibritumomab tiuxetan, idarubicin, ifosfamide, imatinib mesylate, interferon alfa 2a, ipilimumab, ixabepilone, lapatinib ditosylate, lenalidomide, letrozole, leucovorin, leuprolide acetate, levamisole, lomustine, mechlorethamine, melphalan, methotrexate, mitomycin C, mitoxantrone, nelarabine, nilotinib, oxaliplatin, paclitaxel, paclitaxel protein-bound particle, pamidronate, panitumumab, pegaspargase, peginterferon alfa-2b, pemetrexed disodium, pentostatin, raloxifene, rituximab, sorafenib, streptozocin, sunitinib maleate, tamoxifen, temsirolimus, teniposide, thalidomide, toremifene, tositumomab, trastuzumab, tretinoin, uramustine, vandetanib, vemurafenib, vinorelbine, zoledronate.

Therapeutic agents can also include, but are not limited to, acetazolamide, acetohexamide, acrivastine, alatrofloxacin, albuterol, alclofenac, aloxiprin, alprostadil, amodiaquine, amphotericin, amylobarbital, aspirin, atorvastatin, atovaquone, baclofen, barbital, benazepril, bezafibrate, bromfenac, bumetanide, butobarbital, candesartan, capsaicin, captopril, cefazolin, celecoxib, cephadrine, cephalexin, cerivastatin, cetrizine, chlorambucil, chlorothiazide, chlorpropamide, chlorthalidone, cinoxacin, ciprofloxacin, clinofibrate, cloxacillin, cromoglicate, cromolyn, dantrolene, dichlorophen, diclofenac, dicloxacillin, dicumarol, diflunisal, dimenhydrinate, divalproex, docusate, dronabinol, enoximone, enalapril, enoxacin, enrofloxacin, epairestat, eposartan, essential fatty acids, estramustine, ethacrynic acid, ethotoin, etodolac, etoposide, fenbufen, fenoprofen, fexofenadine, fluconazole, flurbiprofen, fluvastatin, fosinopril, fosphenytoin, fumagillin, furosemide, gabapentin, gemfibrozil, gliclazide, glipizide, glybenclamide, glyburide, glimepiride, grepafloxacin, ibufenac, ibuprofen, imipenem, indomethacin, irbesartan, isotretinoin, ketoprofen, ketorolac, lamotrigine, levofloxacin, levothyroxine, lisinopril, lomefloxacin, losartan, lovastatin, meclofenamic acid, mefenamic acid, mesalamine, methotrexate, metolazone, montelukast, nalidixic acid, naproxen, natamycin, nimesulide, nitrofurantoin, non-essential fatty acids, norfloxacin, nystatin, ofloxacin, oxacillin, oxaprozin, oxyphenbutazone, penicillins, pentobarbital, perfloxacin, phenobarbital, phenytoin, pioglitazone, piroxicam, pramipexol, pranlukast, pravastatin, probenecid, probucol, propofol, propylthiouracil, quinapril, rabeprazole, repaglinide, rifampin, rifapentine, sparfloxacin, sulfabenzamide, sulfacetamide, sulfadiazine, sulfadoxine, sulfamerazine, sulfamethoxazole, sulfafurazole, sulfapyridine, sulfasalazine, sulindac, sulphasalazine, sulthiame, telmisartan, teniposide, terbutaline, tetrahydrocannabinol, tirofiban, tolazamide, tolbutamide, tolcapone, tolmetin, tretinoin, troglitazone, trovafloxacin, undecenoic acid, ursodeoxycholic acid, valproic acid, valsartan, vancomycin, verteporfin, vigabatrin, and vitamin K-S (II), zafirlukast.

Therapeutic agents can also include, but are not limited to, abacavir, acebutolol, acrivastine, alatrofloxacin, albuterol, albendazole, alprazolam, alprenolol, amantadine, amiloride, aminoglutethimide, amiodarone, amitriptyline, amlodipine, amodiaquine, amoxapine, amphetamine, amphotericin, amprenavir, amrinone, amsacrine, astemizole, atenolol, atropine, azathioprine, azelastine, azithromycin, baclofen, benethamine, benidipine, benzhexol, benznidazole, benztropine, biperiden, bisacodyl, bisanthrene, bromazepam, bromocriptine, bromperidol, brompheniramine, brotizolam, bupropion, butenafine, butoconazole, cambendazole, camptothecin, carbinoxamine, cephadrine, cephalexin, cetrizine, cinnarizine, chlorambucil, chlorpheniramine, chlorproguanil, chlordiazepoxide, chlorpromazine, chlorprothixene, chloroquine, cimetidine, ciprofloxacin, cisapride, citalopram, clarithromycin, clemastine, clemizole, clenbuterol, clofazimine, clomiphene, clonazepam, clopidogrel, clozapine, clotiazepam, clotrimazole, codeine, cyclizine, cyproheptadine, dacarbazine, darodipine, decoquinate, delavirdine, demeclocycline, dexamphetamine, dexchlorpheniramine, dexfenfluramine, diamorphine, diazepam, diethylpropion, dihydrocodeine, dihydroergotamine, diltiazem, dimenhydrinate, diphenhydramine, diphenoxylate, diphenylimidazole, diphenylpyraline, dipyridamole, dirithromycin, disopyramide, dolasetron, domperidone, donepezil, doxazosin, doxycycline, droperidol, econazole, efavirenz, ellipticine, enalapril, enoxacin, enrofloxacin, eperisone, ephedrine, ergotamine, erythromycin, ethambutol, ethionamide, ethopropazine, etoperidone, famotidine, felodipine, fenbendazole, fenfluramine, fenoldopam, fentanyl, fexofenadine, flecainide, flucytosine, flunarizine, flunitrazepam, fluopromazine, fluoxetine, fluphenthixol, fluphenthixol decanoate, fluphenazine, fluphenazine decanoate, flurazepam, flurithromycin, frovatriptan, gabapentin, granisetron, grepafloxacin, guanabenz, halofantrine, haloperidol, hyoscyamine, imipenem, indinavir, irinotecan, isoxazole, isradipine, itraconazole, ketoconazole, ketotifen, labetalol, lamivudine, lanosprazole, leflunomide, levofloxacin, lisinopril, lomefloxacin, loperamide, loratadine, lorazepam, lormetazepam, lysuride, mepacrine, maprotiline, mazindol, mebendazole, meclizine, medazepam, mefloquine, melonicam, meptazinol, mercaptopurine, mesalamine, mesoridazine, metformin, methadone, methaqualone, methylphenidate, methylphenobarbital, methysergide, metoclopramide, metoprolol, metronidazole, mianserin, miconazole, midazolam, miglitol, minoxidil, mitomycins, mitoxantrone, molindone, montelukast, morphine, moxifloxacin, nadolol, nalbuphine, naratriptan, natamycin, nefazodone, nelfinavir, nevirapine, nicardipine, nicotine, nifedipine, nimodipine, nimorazole, nisoldipine, nitrazepam, nitrofurazone, nizatidine, norfloxacin, nortriptyline, nystatin, ofloxacin, olanzapine, omeprazole, ondansetron, omidazole, oxamniquine, oxantel, oxatomide, oxazepam, oxfendazole, oxiconazole, oxprenolol, oxybutynin, oxyphencyclimine, paroxetine, pentazocine, pentoxifylline, perchlorperazine, perfloxacin, perphenazine, phenbenzamine, pheniramine, phenoxybenzamine, phentermine, physostigmine, pimozide, pindolol, pizotifen, pramipexol, pranlukast, praziquantel, prazosin, procarbazine, prochlorperazine, proguanil, propranolol, pseudoephedrine, pyrantel, pyrimethamine, quetiapine, quinidine, quinine, raloxifene, ranitidine, remifentanil, repaglinide, reserpine, ricobendazole, rifabutin, rifampin, rifapentine, rimantadine, risperidone, ritonavir, rizatriptan, ropinirole, rosiglitazone, roxaditine, roxithromycin, salbutamol, saquinavir, selegiline, sertraline, sibutramine, sildenafil, sparfloxacin, spiramycins, stavudine, sulconazole, sulphasalazine, sulpiride, sumatriptan, tacrine, tamoxifen, tamsulosin, temazepam, terazosin, terbinafine, terbutaline, terconazole, terfenadine, tetramisole, thiabendazole, thioguanine, thioridazine, tiagabine, ticlopidine, timolol, tinidazole, tioconazole, tirofiban, tizanidine, tolterodine, topotecan, toremifene, tramadol, trazodone, triamterene, triazolam, trifluoperazine, trimethoprim, trimipramine, tromethamine, tropicamide, trovafloxacin, vancomycin, venlafaxine, vigabatrin, vinblastine, vincristine, vinorelbine, vitamin K₅, vitamin K₆, vitamin K₇, zafirlukast, zolmitriptan, zolpidem, and zopiclone. Other therapeutic agents are also applicable, as would be recognized by one or ordinary skill in the art based on the present disclosure.

### 4. Examples

Exemplary embodiments of the present disclosure include assessing the effects of a therapeutic agent that is a PCSK9 inhibitor, as described herein. Proprotein convertase subtilisin kexin type 9 (PCSK9), is a protein (serine protease) synthesized and secreted mainly by the liver, which binds to hepatic LDL receptors. It regulates plasma LDL-C levels by diverting cell surface LDL receptors to lysosomes for degradation. In so doing, PCSK9 prevents the normal recycling of LDL receptors back to the cell surface. This process results in reduced LDL receptor density, decreased clearance of LDL-C, and, consequently, accumulation of LDL-C in the circulation. Thus, PCSK9 levels tend to correlate directly with LDL-C levels. In animal models, it is known that mutations that increase PCSK9 activity cause hypercholesterolemia and coronary heart disease (CHD); mutations that inactivate PCSK9 lower LDL levels and reduce CHD. PCSK9 inhibitors are therefore considered attractive potential therapeutic agents for FH, including HoFH. In accordance with the various embodiments of the present disclosure, PCSK9 inhibitors include anti-PCSK9 antibodies (e.g., antibodies that bind to PCSK9 and prevent it binding to liver LDL receptors), such as, but not limited to, evolocumab, alirocumab, bococizumab, RG7652, LY3015014, and LGT-209. PCSK9 inhibitors include the RNAi oligonucleotide ALN-PCSsc and the pegylated adnectin, BMS-962476.

a. Inflammation Signatures

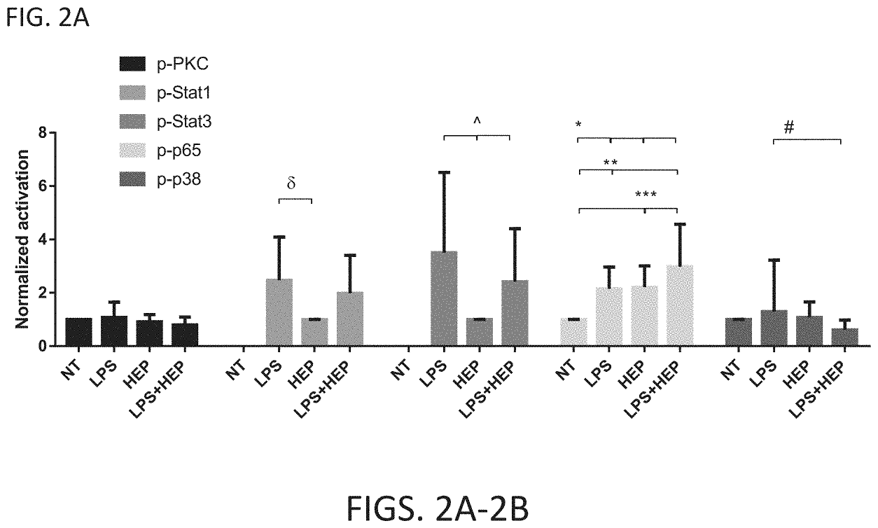

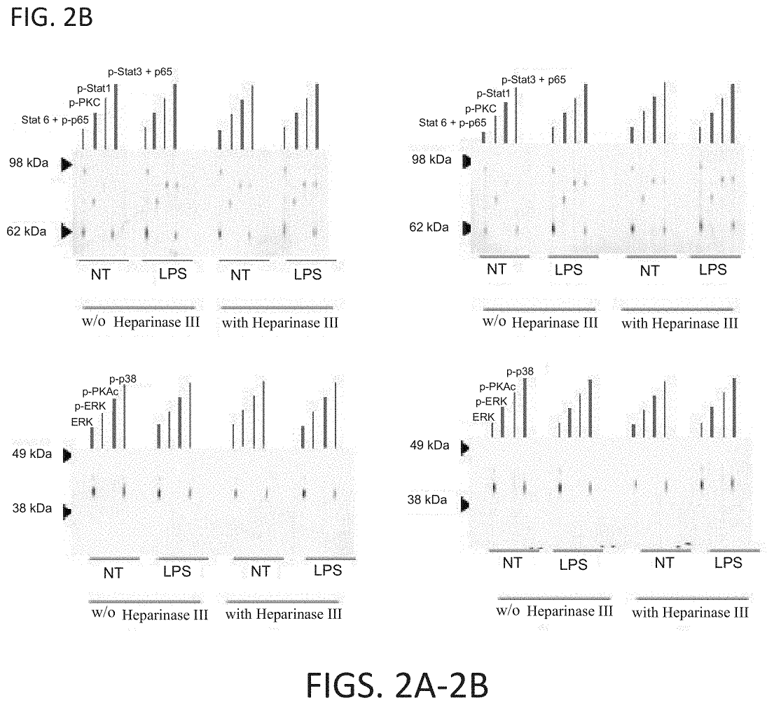

FIGS. 2A-2B include representative graphs of inflammation signature quantification for PBMC lysates treated with LPS, Heparinase III and LPS+Heparinase III for 2 hours, according to one embodiment of the present disclosure. As shown in FIG. 2A, PBMCs were treated with 1 μg/ml LPS, and 1.5 U/mL of Heparinase III. About 1 μg of protein were run and probed for Stat6 (loading control), p-PKC, p-Stat1, p-Stat3, p44/42 (loading control), p-p44/42, p-PKAc, and p-p38 using microfluidic immunoblotting platform. For statistical analysis two-way Anova, Dunnett's post-hoc test was used. For p-p65*, ** and *** denotes statistical difference between treatments, and NT, LPS and Heparinase III; δ represents statistical difference between LPS and Heparinase III treatment for p-Stat1; \hat{ } represents statistical difference between LPS, and Heparinase III and LPS Heaprinase III cotreatment for p-Stat3; and # represents statistical difference between LPS and LPS+Heparinase III treatment for p-p38. Heparinase III treatment leads to activation of Stat1/3, although not as much as LPS; treatment also attenuates LPS Stat1/3 response when done together. Heparinase III treatment increases p65 activation though similar to LPS response. Interestingly, cotreatment of Heparinase and LPS increases p65 activation to a level higher than either treatment when done separately. FIG. 2B shows a representative 2 hr microfluidics immunoblot, according to one embodiment of the present disclosure.

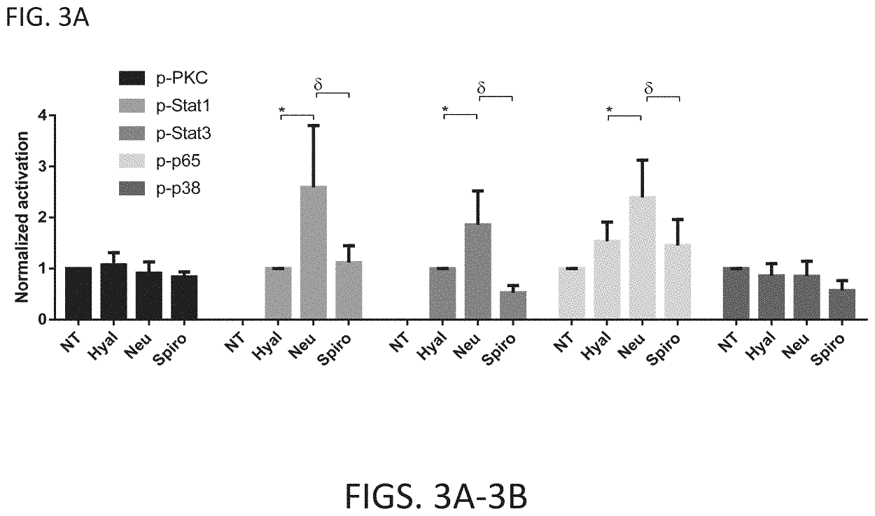

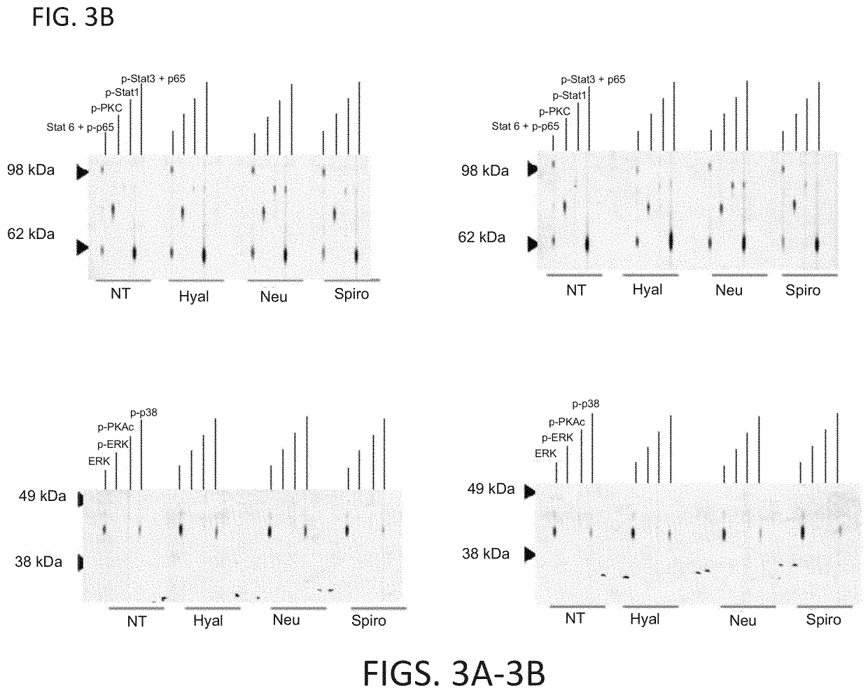

FIGS. 3A-3B include representative graphs of inflammation signature quantification for PBMC lysates treated with Hyaluronidase (Hyal), Neuraminidase (Neu) and Spironolactone for 2 hours, according to one embodiment of the present disclosure. As shown in FIG. 3A, PBMCs were treated with 1 μg/ml LPS, and 1.5 U/mL of Heparinase III. About 1 μg of protein were run and probed for Stat6 (loading control), p-PKC, p-Stat1, p-Stat3, p44/42 (loading control), p-p44/42, p-PKAc, and p-p38 using microfluidic immunoblotting platform. For statistical analysis two-way Anova, Dunnett's post-hoc test was used. For p-p65*, ** and *** denotes statistical difference between treatments, and NT, LPS and Heparinase III; δ represents statistical difference between LPS and Heparinase III treatment for p-Stat1; \hat{ } represents statistical difference between LPS, and Heparinase III and LPS Heaprinase III cotreatment for p-Stat3; and # represents statistical difference between LPS and LPS+Heparinase I treatment for p-p38. Heparinase III treatment leads to activation of Stat1/3, although not as much as LPS; treatment also attenuates LPS Stat1/3 response when done together. Heparinase III treatment increases p65 activation though similar to LPS response. Interestingly, cotreatment of Heparinase and LPS increases p65 activation to a level higher than either treatment when done separately. FIG. 3B shows a representative 2 hr microfluidics immunoblot, according to one embodiment of the present disclosure.

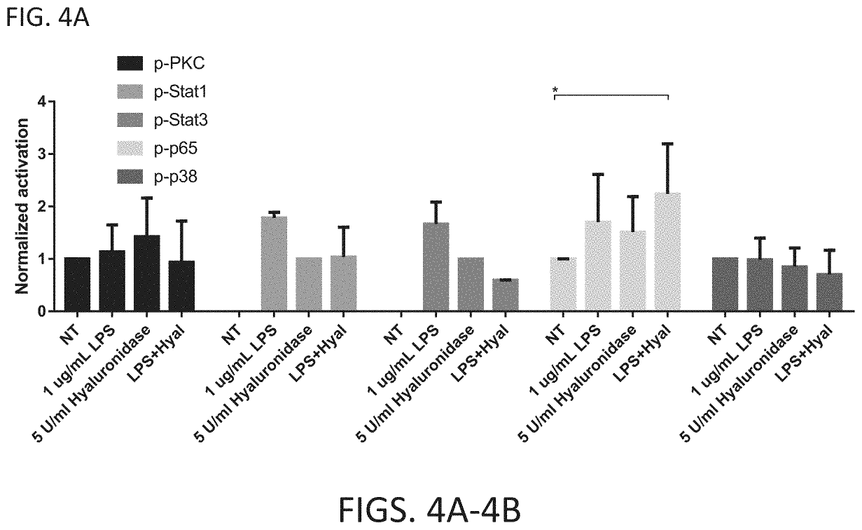

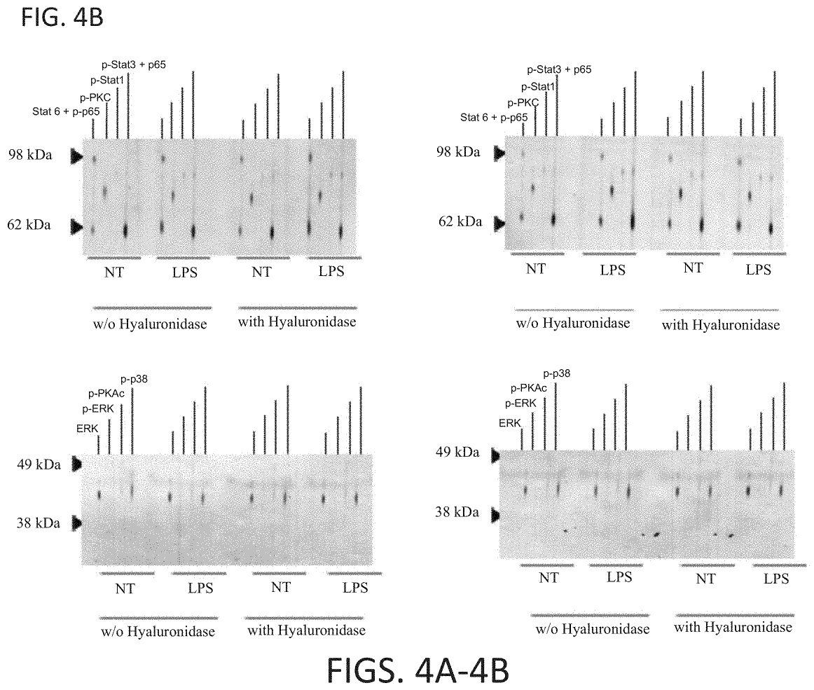

FIGS. 4A-4B include representative graphs of inflammation signature quantification for PBMC lysates treated with LPS, Hyaluronidase and LPS+Hyaluronidase for 2 hours, according to one embodiment of the present disclosure. As shown in FIG. 4A, PBMCs were treated with 2 U/mL of Hyaluronidase, 0.25 U/mL Neurominidase and 100 μM Spironolactone. 1 μg of protein were run and probed for Stat6 (loading control), p-PKC, p-Stat1, p-Stat3, p44/42 (loading control), p-p44/42, p-PKAc, and p-p38 using microfluidic immunoblotting platform. For statistical analysis two way Anova, Dunnett's post-hoc test was used. * denotes statistical difference between Hyal and Neu treatments, while δ represents statistical difference between Neu and Spiro treatment. Neuraminidase treatment increases Stat1/3 and p65 activation as compare to Hyal and Spiro. FIG. 3B shows a representative 2 hr microfluidics immunoblot, according to one embodiment of the present disclosure.

b. Evolocumab

In one embodiment of the present disclosure, the effects of evolocumab upon biocellular markers potentially altered by PCSK9 inhibition in a population of type 2 diabetes patients with cardiac microvascular dysfunction are investigated. For example, the acute and short-term effects of PCSK9 inhibition with evolocumab on biocellular markers of inflammation, endothelial function and rheology are determined, and corresponding data is used to support a clinical trial to assess the role of PCSK9 inhibition in type 2 diabetes patients with cardiac microvascular dysfunction. These studies aid in defining how PCSK9 inhibition alters circulating monocyte subsets, and aid in the evaluation of how PCSK9 inhibition modulates inflammatory signaling pathway activation and transcriptional programs in response to TLRs.

c. Methods

Biocellular Marker Assessments. The impact on evolocumab on biomarkers of endothelial function is evaluated. Biomarkers of oxidative stress (MDA), inflammation (MPO), cytokines (IL-6, IL-18 and TNF-α) and vascular endothelial activation (PECAM, ICAM, VCAM and alpha5/beta 3 activation) are also assessed.

Sample Size Estimation. Embodiments of the present disclosure were designed to investigate the effects of PCSK9 inhibition in a population of patients with type 2 diabetes and microvascular dysfunction. Although there is a paucity of studies that examine drug effects using molecular proteomics, initial data based on the mean and standard deviation of the phospho-p65 (NF-kB component) in healthy control PBMC stimulated with LPS (1 μg/ml) for 2 hours, a power calculation (using 2-tailed students t-test) indicate that this model would be able to distinguish a relative signal difference of 20% (for PCSK9 inhibitor treated patients) with approximately 38 patients assuming an a-value of 0.05 and a statistical power of 80%.

Various PBMC phenotyping approaches can include flow cytometry profiling of PBMC subsets. For example, a significant difference was found between circulating PBMC subsets in HF patients treated with diuretics during a short-time period, suggesting that circulating PBMCs are used to interrogate both local and systemic inflammation in clinical populations; therefore, flow cytometry is effective for profiling PBMC subsets.

Statistical Analysis. To address both early and late changes in biocellular responses as a result of PCSK9 inhibition, changes between week 2 and week 12 are compared with baseline levels. Week 2 is compared with week 12 to identify how the expected rheological changes may impact biocellular responses. Microfluidic protein immunoblots are analyzed using ImageJ software. If proteomic data is normally distributed, statistical significance is determined by one-factor ANOVA with Bonferroni correction and Student's t-test with p-value <0.05 considered statistically significant. If proteomic data is not normally distributed, statistical significance are determine using Mann-Whitney non-parametric test.

Primary efficacy analysis. The primary efficacy comparison between groups is by means of linear regression (ANCOVA model). The point estimate of the effect and 95% confidence interval are obtained, after adjustment for the baseline values. The primary analysis is ITT.

Data Management. All study data is stored in an electronic database system, created and managed by Mount Sinai's International Center for Health Outcomes and Innovation Research. Study personnel needing access will have their own Login/Password. Access to clinical study information is based on individuals' roles and responsibilities. The application provides hierarchical user permission for data entry, viewing, and reporting options. The application is designed to be in full compliance with International Conference on Harmonization and Good Clinical Practices (ICH-GCP), the FDA's Code of Federal Regulations Number 21 Part 11 Electronic Record and Electronic Signatures, the FDA's “Guidance: Computerized Systems Used in Clinical Trials, and the Privacy Rule of the Health Insurance Portability and Accountability Act of 1996 (HIPAA).”

Tables 1 and 2 below provide exemplary timelines for clinical assessments.

TABLE 1
Study Timeline Icahn School of Medicine at Mount Sinai (16 Subjects)

| | | Timelines | | | | | | | |
|---|---|---|---|---|---|---|---|---|---|
| | | Year 1 | | | | Year 2 | | | |
| Task | Month | 0-3 | 3-6 | 6-9 | 9-12 | 12-15 | 15-18 | 18-21 | 21-24 |

Study Startup
Patient Recruitment
Baseline Biocellular
Assessment (Study
Visit 2/Day 0)
ACUTE Term Biocellular
Assessment (Study
Visit 3/Week 2)
SHORT-Term Biocellular
Assessment (Study
Visit 6/Week 12)
Biocellular Assessment
Analysis
Statistical Analysis
Manuscript Publication

TABLE 2
St Michael's Hospital (24 Subjects)

| | | Timelines | | | | | | | |
|---|---|---|---|---|---|---|---|---|---|
| | | Year 1 | | | | Year 2 | | | |
| Task | Month | 0-3 | 3-6 | 6-9 | 9-12 | 12-15 | 15-18 | 18-21 | 21-24 |

Study Startup
Patient Recruitment
Baseline Biocellular
Assessment (Study
Visit 2/Day 0)
ACUTE Term Biocellular
Assessment (Study
Visit 3/Week 2)
SHORT-Term Biocellular
Assessment (Study
Visit 6/Week 12)
Biocellular Assessment
Analysis
Statistical Analysis
Manuscript Publication

d. Study Subjects

Subject Eligibility. This study can only fulfill its objectives if appropriate subjects are enrolled. In addition to the eligibility criteria listed below, all relevant medical and non-medical considerations are taken into account when deciding whether an individual subject is suitable to enter this particular study.

Inclusion Criteria for Biocellular Marker Study. Inclusion criteria include the following: subjects ≥18 years of age at signing of informed consent; stable CAD who have undergone prior PCI or CABG greater than 6 months from screening and in whom no further revascularization is planned within the duration of the study at the time of randomization; clinical diagnosis of type 2 diabetes according to ADA/CDA guidelines; subject on stable dose of maximally-tolerated statin therapy for ≥4 weeks prior to screening and LDL-C≥70 mg/dL. For subjects whose maximally tolerated dose of statin is no type or dose (i.e., determined to be statin intolerant by primary investigator), background lipid-lowering therapy is not required. Fasting triglycerides ≤400 mg/dL (4.52 mmol/L) by central laboratory at screening. Willing and able to comply with scheduled visits, treatment plan, laboratory tests and other trial procedures. Abnormal urinary Albumin Creatinine Ratio (ACR) as defined by an ACR>2. Subject tolerates screening placebo injection.

Exclusion Criteria for Biocellular Marker Study. Exclusion criteria include the following: personal or family history of hereditary muscular disorders; NYHA III or IV heart failure, or last know left ventricular ejection fraction (LVEF)<30%; uncontrolled serious cardiac arrhythmia defined as recurrent and highly symptomatic ventricular tachycardia, atrial fibrillation with rapid ventricular response, or supraventricular tachycardia that are not controlled by medications, in the past 3 months prior to randomization; myocardial infarction, unstable angina, percutaneous coronary intervention (PCI), coronary artery graft (CABG) or stroke within 3 months prior to randomization; planned cardiac surgery or revascularization; moderate to severe renal dysfunction, defined as an estimated glomerular filtration rate (eGFR)<30 mL/min/1.73 m² at screening; type 1 diabetes, poorly controlled type 2 diabetes (HbA1c>10%), newly diagnosed type 2 diabetes (within 6 months of randomization), or laboratory evidence of diabetes during screening (fasting serum glucose ≥126 mg/dL [7.0 mmol/L] or HbA1c≥6.5% without prior diagnoses of diabetes; uncontrolled hypertension defined as sitting systolic blood pressure (SBP)>160 mmHg or diastolic BP (DBP)>100 mmHg; subject who has taken a cholesterol ester transfer protein (CETP) inhibitor in the last 12 months prior to LDL-C screening, such as: anacetrapib, dalcetrapib or evacetrapib; treatment in the last 3 months prior to LDL-C screening with any of the following drugs: systemic cyclosporine, systemic steroids (e.g., IV, intramuscular [IM], or PO) (Note: hormone replacement therapy is permitted), vitamin A derivatives and retinol derivatives for the treatment of dermatologic conditions (e.g., Accutane); (Note: vitamin A in a multivitamin preparation is permitted). Topical retinol prescription and non-prescription derivatives or creams are permitted; uncontrolled hypothyroidism or hyperthyroidism as defined by thyroid stimulating hormone (TSH)<1.0 time the lower limit of normal or >1.5 times the ULN, respectively, at screening; potential subjects with TSH<1.0 time the lower limit of normal due to thyroid replacement therapy is not considered an exclusion; active liver disease or hepatic dysfunction, defined as aspartate aminotransferase (AST) or alanine aminotransferase (ALT)>3 times the ULN as determined by central laboratory analysis at screening; known active infection or major hematologic, renal, metabolic, gastrointestinal or endocrine dysfunction in the judgment of the investigator; diagnosis of deep vein thrombosis or pulmonary embolism within 3 months prior to randomization; unreliability as a study participant based on the investigator's (or designee's) knowledge of the subject (e.g., alcohol or other drug abuse; currently enrolled in another investigational device or drug study, or less than 30 days since ending another investigational device or drug study(s), or receiving other investigational agent(s); female subject who has either (1) not used at least 1 highly effective method of contraception for at least 1 month prior to screening or (2) is not willing to use such a method during treatment and for an additional 15 weeks after the end of treatment, unless the subject is sterilized or postmenopausal; menopause is defined as: 12 months of spontaneous and continuous amenorrhea in a female >55 years old or 12 months of spontaneous and continuous amenorrhea with a follicle-stimulating hormone (FSH) level >40 IU/L (or according to the definition of “postmenopausal range” for the laboratory involved) in a female <55 years old unless the subject has undergone bilateral oophorectomy; highly effective methods of birth control include: not having intercourse or using birth control methods that work at least 99% of the time when used correctly and include: birth control pills, shots, implants, or patches, intrauterine devices (IUDs), tubal ligation/occlusion, sexual activity with a male partner who has had a vasectomy, condom or occlusive cap (diaphragm or cervical/vault caps) used with spermicide; subject who is pregnant or breast feeding, or planning to become pregnant during treatment and/or within 15 weeks after the end of treatment; subject who has previously received an approved PCSK9 inhibitor or any other investigational therapy to inhibit PCSK9; subject who has any kind of disorder that, in the opinion of the investigator, may compromise the ability of the subject to give written informed consent and/or to comply with all required study procedures; malignancy except non-melanoma skin cancers, cervical or breast ductal carcinoma in situ within the last 5 years; subject who has known sensitivity to any of the products or components to be administered during dosing; subject who is likely to not be available to complete all protocol-required study visits or procedures, and/or to comply with all required study procedures to the best of the subject and investigator's knowledge; history or evidence of any other clinically significant disorder, condition or disease (with the exception of those outlined above) that, in the opinion of the principal investigator would pose a risk to subject safety or interfere with the study evaluation, procedures or completion; use of ranolazine either prior to screening or planned addition of ranolazine during the course of the study; frequent episodes (>4/month) of mild-moderate hypoglycemia, as defined by the Canadian Diabetes Association 2008 Clinical Practice Guidelines for the Prevention and Management of Diabetes; any episode of severe hypoglycemia within the past 3 months, as also defined by the Canadian Diabetes Association 2008 Clinical Practice Guidelines for the Prevention and Management of Diabetes; blood donation 4 weeks prior to screening, or stated intention to donate blood or blood products during the period of the study or within one month following completion of the study; subjects who have participated in other studies within 30 days prior to screening, or have five times the plasma half-life (if known) of the investigational drug, whichever is longer; BMI>40 kg/m².

e. Study Overview

This is a multi-center, double-blind, randomized, placebo-controlled, parallel group Phase IV study with two treatment arms: evolocumab SC 420 mg/dL QM or matching placebo. The population includes men and women of non-child-bearing potential with documented CAD and type 2 diabetes.

Approximately 40 subjects are screened. Subjects are followed for 12 weeks during the treatment phase, maintaining the double-blind throughout. Assessments of acute and short-term effects of PCSK9 inhibition with evolocumab on biocellular markers of endothelial function are measured at baseline, Week 2, and Week 12. Safety assessments are undertaken at each study visit. Subjects whose maximally tolerated dose of statin is no type or dose (i.e., determined to be statin intolerant by primary investigator) should be randomized fairly between 2 treatment arms and across 2 sites to prevent selection bias.

Subjects should continue on their stable dose of anti-hyperglycaemic therapies during the course of the study. However, it is important that the haemoglobin A1c (HbA1c) does not rise above 10% and that the patient does not develop severe hypoglycaemia or frequent episodes of mild-moderate hypoglycaemia, as defined by the Canadian Diabetes Association 2008 Clinical Practice Guidelines for the Prevention and Management of Diabetes.

Randomization Procedure. A randomization list is produced using a validated system that automates the random assignment of treatment arms to randomization numbers in the specified ratio stratified between 2 sites. Randomization is centralized, web-based and generated by the Applied Health Research Centre (AHRC) at the Li Ka Shing Knowledge Institute of St. Michael's Hospital. Randomization is stratified by center using random permuted blocks of varying sizes. Randomization is web-based through the electronic case report forms (eCRFs), which is accessed by each site. At Visit 2, eligible subjects are given the lowest available randomization number. This number assigns the patient to one of the treatment arms.

Study Medication. The blinded study medication consists of: Evolocumab 420 mg SC QM; and Matching Placebo SC QM.

Blinding. The identity of the treatments are concealed by the use of matching placebo to the study drug that are identical in packaging, labeling, appearance and schedule of administration (Amgen).

Premature Interruption of Study Treatment. Subjects have the right to discontinue study treatment for any reason. However, unless they withdraw consent and are no longer willing to participate, they should be followed by telephone calls for the remainder of the trial and all serious events must be reported, regardless of whether or not the event is of cardiovascular origin or occurs under the care of another physician or institution. In case of a temporary interruption of study treatment, all efforts should be made to re-institute study treatment as soon as the clinical condition of the subject has stabilized based on the judgment of the Investigator.

Withdrawal of Consent. Subjects may decide to fully or partially withdraw their consent to participate in the study. At that point, it should be established, whether the withdrawal is related to study treatment, further assessments, or any further involvement in the study. If the withdrawal is primarily related to study treatment or specific assessments, subjects should be encouraged to continue follow-up by telephone calls and to attend all other subsequent study assessments. Reports on serious events should be collected until completion of the study for all withdrawals, unless the subject objects to such follow-up.

Concomitant Therapy. Evolocumab has a low potential for drug interactions with commonly co-prescribed medications for patients with type 2 diabetes. Subjects will continue their standard of care treatment. Due to the impact of anti-anginal agents such as, but not limited to ranolazine, upon microvascular function, the use of ranolazine is an exclusion criterion and the addition of ranolazine is be permitted during the duration of the study. All other anti-anginal medicines are permitted.

Schedule of Study Procedures. Screening assessments and study procedures outlined in this section and in Table 3 (below) are performed after obtaining written informed consent. All on-study visits and dosing should be scheduled from Day 1 (first IP administration). For example, the Week 2 visit is two calendar weeks after the study Day 1 visit, which corresponds to study Week 2. When it is not possible to perform the study visit at the specified time point, the visit should be performed within the visit window specified in Table 3. If a study visit is missed or late, including visits outside the visit window, subsequent visits should resume on the original visit schedule. Missed assessments at prior visits should not be duplicated at subsequent visits. With the exception of screening, all study procedures for a visit should be completed on the same day if possible.

| | Schedule of Assessments | | | | | |
|---|---|---|---|---|---|---|
| | | Day 1/ | | | | |
| | Screening | Randomization | Week 2 | Week 4 | Week 8 | Week 12 |
| | Visit Number | | | | | |
| | | 2 | 3 | 4 | 5 | 6 |
| Visit Window | 1 | ±7-12 Days | ±4 Days | ±7 Days | ±7 Days | ±7 Days |
| General Procedures | | | | | | |
| Informed consent | X | | | | | |
| Demographics & Medical history | X | | | | | |
| Vital signs (including HR, sitting and standing | X | X | X | X | X | X |
| blood pressure as the average of 3 | | | | | | |
| measurements using standardized techniques | | | | | | |
| Review for AEs/SAEs/PEPs | X | X | X | X | X | X |
| Concomitant therapy; medication compliance reminder | X | X | X | X | X | X |
| Dietary instruction | X | X | X | X | X | X |
| Physical and neurological examination | X | | | | | |
| Body height, waist circumference | X | X | | | | X |
| Body weight | X | X | | | | X |
| 12 lead ECG triplicate measurements | X | | | | | X |
| Seattle Angina Questionaire | X | X | X | X | X | X |
| Laboratory Analyses: | X | X | X | X | X | X |
| 1) Whole Blood Viscosity Analysis (Visits 2-6 only) | | | | | | |
| 2) Lipoprotein Subclass Analysis (Visits 2-6 only, | | | | | | |
| Lipid Panel at Visit 1) | | | | | | |
| 3) Urinary Albumin Creatinine Ratio (ACR) | | | | | | |
| 4) Complete Blood Count | | | | | | |
| 5) Fibrinogen | | | | | | |
| 6) HbA1_{C} (Visit 1 only) | | | | | | |
| 6) Urine Pregnancy Test (Visit 1 only) | | | | | | |
| Laboratory Analyses: | | X | X | | | X |
| 1) Biocellular Marker Analysis | | | | | | |
| Screening Placebo Run-In SC Injections - 3 total | X | | | | | |
| (Provided by Sponsor) | | | | | | |
| Randomizaton including Subject Instructions on Safety, | | X | | | | |
| IP adherance, Storage, Daily Log Time of when IP is | | | | | | |
| taken (includes weekly reminder calls to ensure | | | | | | |
| medication compliance) | | | | | | |
| Investigational Product | | | | | | |
| SC IP Amninistration, QM | | X | | X | X | |

Visit 1: Screening. Written informed consent; Demographics: Medical history; Vital signs, including HR, sitting and standing blood pressure as the average of 3 measurements using standardized techniques; Review for AEs/SAEs/PEPs; Concomitant therapy; Dietary instruction; medication compliance reminder; Physical and neurological examination; Body height, waist circumference; Body weight; Seattle Angina Questionnaire; Laboratory assessments: hematology, blood chemistry, Liver Function Tests (LFTs), creatinineeGFR, HbA_{1C}, local lipid panel, urinalysis, urine pregnancy test (if female of child bearing potential); Screening placebo injections and 30 minute post-injections observation

Visit 2 (Randomization) 7-12 days after Screening. Review for AEs/SAEs/CV events; Concomitant therapy; 12 lead ECG triplicate measurements; Dietary instruction; medication compliance reminder: Randomization; Seattle Angina Questionnaire; Laboratory assessments: hematology, blood chemistry, Liver Function Tests (LFTs), fibrinogen creatinine/eGFR, HbA_{1C}, urinalysis, whole blood viscosity analysis, NMR lipoprotein subclass analysis, endothelial and inflammatory biocellular markers analysis; SC IP administration, QM (in-clinic) and 30 minute post-injections observation.

Visit 3 (Week 2) 4 days. Review for AEs/SAEs/CV events; Concomitant therapy; Dietary instruction; medication compliance reminder; Seattle Angina Questionnaire; Laboratory assessments: hematology, blood chemistry, Liver Function Tests (LFTs), fibrinogen creatinine/eGFR, whole blood viscosity analysis, NMR lipoprotein subclass analysis, endothelial and inflammatory biocellular markers analysis.

Visit 4 (Week 4)+7 days. Review for AEs/SAEs/CV events; Concomitant therapy; Dietary instruction; medication compliance reminder; Seattle Angina Questionnaire; Laboratory assessments: blood chemistry, Liver Function Tests (LFTs), creatinine/eGFR; SC IP administration, QM (in-clinic) and 30-minute post-injections observation

Visit δ (Week 8)+7 days. Review for AEs/SAEs/CV events; Concomitant therapy; Dietary instruction; medication compliance reminder: Seattle Angina Questionnaire: Laboratory assessments: blood chemistry, Liver Function Tests (LFTs), creatinine/eGFR; SC IP administration, QM (in-clinic) and 30-minute post-injections observation.

Visit 6 (Week 12) 17 days. Body height, waist circumference; Body weight; 12 lead ECG triplicate measurements; Review for AEs/SAEs/CV events; Concomitant therapy; Dietary instruction; medication compliance reminder: Seattle Angina Questionnaire; Laboratory assessments: hematology, blood chemistry, Liver Function Tests (LFTs), fibrinogen creatinineeGFR, urinalysis, whole blood viscosity analysis, NMR lipoprotein subclass analysis, endothelial and inflammatory biocellular markers analysis

f. Clinical Procedures and Safety Evaluations

Informed Consent. Once deemed appropriate candidates for the study, the subject is informed of the possibility of study participation. The benefits and risks of participating in the study is explained to the subject, and the subject is provided an opportunity to read the informed consent form and ask any questions he/she may have. Prior to conducting any study-related procedures, the subject should provide consent to participate by signing the Institutional Review Board/Research Ethics Board (IRB/REB) approved consent form.

Demography. This includes age, sex and race.

Standard 12-lead ECG. A 12-lead ECG is performed at Screening to obtain triplicate measurements.

Medical History. Data is collected from subjects at baseline consisting of demographics, history, previous cardiac investigations, previous cardiac history, and current medication.

Physical Examination. A routine physical examination is required at Visit 1. Any abnormality should be recorded.

Vital Signs. Vital signs including blood pressure and heart rate are recorded at the intervals indicated on the schedule of study procedures in section 13.

Body weight, Height, and Waist Circumference. Body weight, height, and waist circumference are recorded at Visit 1 (Screening), Visit 2 (Randomization/Baseline), and Visit 6 (Week 12).

Local Laboratory Tests. Local laboratory assessments include hematology, blood chemistry, liver function tests (LFTs), eGFR, HbA_{1C}, and ACR, whole blood viscosity, lipoprotein subclass, and biocellular marker analyses.

Additional Laboratory Tests. Blood is collected for circulating markers of endothelial activation on the same day as the study visit and later analyzed at the central laboratory. In addition, plasma is retained and frozen for future analysis.

Concomitant Therapy. Medications are not altered for the purpose of this trial; however, any medication changes during the study should be recorded, as should those started before randomization and continued thereafter. During study visits, changes in concomitant therapy must be recorded.

Study Drug Accounting. Double-blind study medication may only be dispensed and administered according to the study protocol by authorized personnel, as documented in the investigator's trial staff list. At each visit, compliance is checked and recorded in the case report form.

Adverse Event (AE) Reporting. An adverse event (AE, AEs) is defined as any untoward medical occurrence in a subject who has been administered a pharmaceutical product and which does not necessarily have to have a causal relationship with this treatment. AEs include those reported spontaneously by the subject and those noted incidentally or as observed by the investigator or study staff.

Study staff will assess all AEs that occur during the period from signing of consent until end of study participation and document these in the source documents. Investigators will evaluate any changes in laboratory values and physical symptoms/signs and will determine if the change is clinically significant. If clinically significant and unexpected adverse experiences occur, they are recorded in the case report form (CRF).

Serious Adverse Events (SAEs) and Serious Adverse Drug Reactions (SADRs). Serious Adverse Event is defined as any untoward medical occurrence that at any dose results in: Death; A life-threatening situation (Subject was at risk of death at the time of the event; this does not refer to an event that might have caused death if it was of greater intensity); New in-patient hospitalization or prolongation of an existing hospitalization; Persistent or significant disability or incapacity; Congenital anomaly or birth defect; Important medical events that may not result in death, be life-threatening, or require hospitalization but may jeopardize the subject and may require medical or surgical intervention to prevent one of the above outcomes (based upon appropriate medical judgment), such as allergic bronchospasm requiring intensive treatment in an emergency room or at home, blood dyscrasias or convulsions that do not result in inpatient hospitalization, or the development of drug dependency or drug abuse.

Medical and scientific judgment should be exercised in deciding whether other conditions should also be considered serious, such as important medical events that may not be immediately life-threatening or result in death or hospitalization but may jeopardize the subject or may require intervention to prevent one of the other outcomes listed above. These should also be considered serious. Follow up information regarding SAEs are pursued until the event has resolved (with or without sequelae), until death, or until 30 days after the final study visit (whichever comes first). For any deaths where there is uncertainty about the cause of death, site investigators may request an autopsy if appropriate.

Reporting Serious Adverse Events (A4E). Any SAE, including death due to any cause, which occurs between enrolment and the final follow up visit whether or not related to the study drug, must be reported immediately (within 1 business day of the study site's knowledge of the event). The report will contain as much available information concerning the SAE to enable a report to be produced that satisfies regulatory reporting requirements.

The term “severe” is often used to describe the intensity (severity) of a specific event (as in mild, moderate, or severe pain), the event itself, however, may be of relatively minor medical significance (such as severe headache). By contrast, the term “serious” is used to describe an event based on an event outcome or actions usually associated with events that pose a threat to a subject's life or functioning. Seriousness (not severity) serves as a guide for defining regulatory reporting obligations.

For all collected SAE's, the clinician who examines and evaluates the subject will determine the event's causality based on temporal relationship and his/her clinical judgment. The degree of certainty about causality is graded using the categories below:

Definitely Related: There is clear evidence to suggest a causal relationship, and other possible contributing factors can be ruled out.

Probably Related: There is evidence to suggest a causal relationship, and the influence of other factors is unlikely.

Possibly Related: There is some evidence to suggest a causal relationship. However, the influence of other factors may have contributed to the event.

Unlikely: A clinical event, including an abnormal laboratory test result, whose temporal relationship to drug administration makes a causal relationship improbable and in which other drugs or chemicals or underlying disease provides plausible explanations

Not Related: The SAE is completely independent of study drug administration, and/or evidence exists that the event is definitely related to another etiology.

Pre-existing conditions should be recorded upon patient enrolment (including start date of the condition, and severity—mild, moderate, severe). After the patient signs the informed consent form, any worsening of these conditions would be recorded.

Any new conditions would be recorded including date of onset, date of resolution, severity (mild, moderate, severe, or serious as defined above) and possible relationship to study drug or procedure. As part of the source notes, follow up clinical assessments, laboratory tests, ECGs and diagnostic imaging related to adverse event should be documented.

It is understood that the foregoing detailed description and accompanying examples are merely illustrative and are not to be taken as limitations upon the scope of the disclosure, which is defined solely by the appended claims and their equivalents.

Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the disclosure, may be made without departing from the spirit and scope thereof.

What is claimed is:

1. A method of generating a dynamic molecular signature, the method comprising:
(a) performing an assay on a sample from a subject to detect at least one biocellular marker; and
(b) generating a dynamic molecular signature based on the detection of the at least one biocellular marker from the subject;
wherein the subject has been administered, or is being administered, at least one therapeutic agent.

2. The method of claim 1, wherein the assay is performed using a microfluidic immunoblotting device comprising a flat membrane-contacting surface and a plurality of non-connected parallel microfluidic channels, wherein the entire length of the microfluidic channels are open to the membrane-contacting surface.

3. The method of claim 1 or claim 2, wherein the assay is performed by:
(a) transferring proteins from a sample onto a membrane;
(b) placing a membrane-contacting face of a microfluidic immunoblotting device onto the membrane;
(c) injecting an activating buffer into microfluidic channels of the device;
(d) injecting primary antibody solutions into the microfluidic channels of the device;
(e) detecting binding of antibodies with the antibody solutions to the proteins.

4. The method of any of claims 1 to 3, wherein the assay comprises selecting and isolating a group of cells from the sample prior to generating the dynamic molecular signature.

5. The method of any of claims 1 to 4, wherein the method further comprises assessing one or more effects of the therapeutic agent on gene or protein expression or activation.

6. The method of any of claims 1 to 5, wherein generating a dynamic molecular signature comprises quantifying a level of expression or activation of the at least one biocellular marker in the sample from the subject with reference to a control sample.

7. The method of any of claims 1 to 6, wherein the method further comprises correlating the dynamic molecular signature to one or more results of a clinical assessment of the subject.

8. The method of any of claims 1 to 7, wherein the therapeutic agent is evolocumab, and wherein the dynamic molecular signature represents effects of PCSK9 inhibition on the biocellular markers.

9. The method of any of claims 1 to 8, wherein the at least one biocellular marker comprises a protein involved in MAPK, NFkB, Akt, AMPK, mTOR, Jak-STAT, and PKA signaling pathways.

10. The method of any of claims 1 to 9, wherein the at least one biocellular marker comprises at least one of VCAM-1, ICAM-1, LOX-1, MCP-1 and MIP-1α.

11. The method of any of claims 1 to 10, wherein the at least one biocellular marker comprises at least one protein expressed by a peripheral blood mononuclear cell.

12. The method of any of claims 1 to 11, wherein the at least one biocellular marker comprises at least one protein expressed by a circulating monocyte.

13. The method of any of claims 1 to 12, wherein the method further comprises treating the subject with a therapeutic agent based on the dynamic molecular signature.

14. The method of any of claims 1 to 13, wherein the method further comprises altering an aspect of the treatment based on the dynamic molecular signature.

15. A method of assessing an effect of a therapeutic agent, the method comprising:
(a) performing an assay on a sample from a subject to detect at least one biocellular marker; and
(b) generating a dynamic molecular signature based on the detection of the at least one biocellular marker from the subject;
wherein the subject has been administered, or is being administered, at least one therapeutic agent.

16. A dynamic molecular signature generated from a biological sample from a subject, wherein the signature comprises a detectable level of at least one biocellular marker from the subject across different timepoints or treatment regimens.

17. The molecular signature of claim 16, wherein the at least one biocellular marker comprises a protein involved in MAPK, NFkB, Akt, AMPK, mTOR, Jak-STAT, and PKA signaling pathways.

18. The molecular signature of claim 16, wherein the at least one biocellular marker comprises at least one of VCAM-1, ICAM-1, LOX-1, MCP-1 and MIP-1α.

19. The molecular signature of any of claims 16 to 18, wherein the at least one biocellular marker comprises at least one protein expressed by a peripheral blood mononuclear cell.

20. The molecular signature of any of claims 16 to 19, wherein the at least one biocellular marker comprises at least one protein expressed by a circulating monocyte.

21. The molecular signature of any of claims 16 to 20, wherein the detectable level of the at least one biocellular marker comprises altered expression or activation of the at least one biocellular marker in the sample from the subject with reference to a control sample.
